(19) United States
(12) Patent Application Publication
(10) Pub. No.: US 2010/0298531 A1
(43) Pub. Date: Nov. 25, 2010

(54) PMMA BINDING PEPTIDES

(75) Inventors: Eberhard Schneider, Denkte (DE); Gregor Schurmann, Hannover (DE); Peter Wagner, Braunschweig (DE); Hong Wang, Kennett Square, PA (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: E.I. DU PONT DE NEMOURS AND COMPANY, Wilmington, DE (US)

(21) Appl. No.: 12/778,174

(22) Filed: May 12, 2010

Related U.S. Application Data
(60) Provisional application No. 61/179,824, filed on May 20, 2009.

(51) Int. Cl. C07K 14/00

(52) U.S. Cl. 530/324

(57) ABSTRACT

Peptides are provided that have binding affinity for polymethyl methacrylate (PMMA). The polymethyl methacrylate-binding peptides may be used to prepare peptide-based reagents suitable for use in a variety of applications. The peptide-based reagents may be used to couple benefit agents to a PMMA polymer surface or may be used to couple a benefit agent comprising a PMMA polymer surface to a target surface, such as a body surface.

This application claims the benefit of U.S. Provisional Patent Application No. 61/179,824 filed May 20, 2009.

### FIELD OF THE INVENTION

The invention relates to peptides having affinity for polymethyl methacrylate polymer as well as peptide-based reagents comprising at least one of the present polymethyl methacrylate-binding peptides.

### BACKGROUND OF THE INVENTION

Polymethyl methacrylate resin (PMMA; CAS RN: 9011-14-7) is a clear polymer developed as a glass substitute. It is commonly referred to as acrylic glass or acrylic and marketed under trademarks such as: PLEXIGLAS™, PERSPEX™, ACRYLITE™, ACRYLPLAST™, and LUCITE™. PMMA has several advantages over silicon glass such as lower density, higher impact strength, higher shatter resistance, and a lower processing temperature. PMMA is commonly found in large windows, aquariums, vehicle rear lights, dentures, and paint coatings, to name a few.

The ubiquitous use of PMMA in industry makes it a prime material candidate for a variety of applications in which PMMA comprises some or all of a surface. However, the physical properties of PMMA may be undesirable for certain applications. Under such circumstances a coating may be applied to mask or alter the undesirable property.

One way to alter, mask or enhance certain properties of a PMMA polymer surface is to couple to the surface an agent that provides a desired effect or benefit (a “benefit agent”). However, many benefit agents do not durably adhere to PMMA. As such, there is a need to provide a reagent suitable for enhancing the delivery and/or durability of a benefit agent targeted to a surface comprising PMMA polymer. Preferably, the reagent has a least one portion having strong affinity for PMMA polymer.

Peptide-based reagents can be prepared to couple a benefit agent to a target surface. Peptide sequences that bind to surface comprising PMMA polymer are described by Cunningham et al. in U.S. Patent Application Publication No. 2007/0265431. However, only phage display-identified PMMA-binding peptides are provided by Cunningham et al. It is known that other display techniques, such as mRNA-display, can provide greater sequence diversity that may result in the identification of linear peptides having stronger affinity for the target substrate (Gold, L., (2001) PNAS. 98(9):4825-4826). Additionally, some commercial applications may use peptide reagents comprising a plurality of different PMMA-binding peptides. As such, there is a need to identify additional PMMA-binding peptides having strong affinity for surfaces comprising PMMA polymer.

The problem to be solved is to provide additional PMMA-binding peptides as well as peptide-based reagents suitable for either (1) coupling a first surface comprising PMMA polymer to a benefit agent or (2) coupling a benefit agent comprising PMMA polymer to a second target surface to deliver a benefit to the second target surface.

### SUMMARY OF THE INVENTION

The stated problem has been solved by the identification of PMMA-binding peptides having strong affinity for PMMA polymer. The present PMMA-binding peptides were identified using mRNA-display. One or more of the present PMMA-binding peptides may be used to prepare peptide-based reagents for use in the delivery of at least one benefit agent to a material comprising PMMA resin. One or more of the present PMMA-binding peptides may also be used to form a beneficial film on and/or coupled a benefit agent to a PMMA polymer. The peptide-based reagents may also be used to couple a benefit agent comprising PMMA (the first target surface) to a second target surface. The first and second target surfaces may be the same or different.

Many of the present PMMA-binding peptides share similar structures based on prevalence of conserved sequences identified using mRNA-display. As such, sequences of PMMA-binding peptides sharing significant structural similarity are provided.

In one embodiment, a peptide having affinity for polymethyl methacrylate (i.e. a “PMMA-binding peptide”) is provided, said polypeptide having the general structure (SEQ ID NO: 14):

GSETYLYWSWX₁WLYX₂VVYWPFX₃YX₄WAGM

wherein
i) X₁ is W or C;
ii) X₂ is L or S;
iii) X₃ is W or R; and
iv) X₄ is V or M.

One or more of the present PMMA-binding peptides may be used to prepare peptide reagents. Peptide reagents are also provided having a general structure selected from the group consisting of:

([PBP]ₙ-[L]ₓ-BA-[L]_{y})ₘ; and

([PBP]ₙ-[L]ₓ-TBD-[L]_{y})ₘ

wherein:
i) PBP is a polymethyl methacrylate-binding peptide;
ii) L is a linker molecule;
iii) BA is at least one benefit agent;
iv) TBD is a target binding domain;
v) x and y independently range from 0 to 10;
vi) n=1 to 10; and
vii) m=1 to 10;

wherein the polymethyl methacrylate-binding peptide comprises an amino acid sequence of SEQ ID NO: 14.

In another embodiment, a method for binding a peptide-based reagent to PMMA is provided comprising:

a) providing at least one peptide or peptide reagent comprising SEQ ID NO: 14; and

b) contacting the peptide-based reagent of (a) with a surface comprising PMMA whereby the peptide-based reagent binds to the PMMA.

The present PMMA-binding peptides and/or peptide reagents may be used in personal care compositions to delivery or enhance the durability of a benefit agent to a body surface. As such, a personal care composition comprising one or more of the present PMMA-binding peptides and/or peptide reagents is also provided.

### BRIEF DESCRIPTION OF THE BIOLOGICAL SEQUENCES

The following sequences comply with 37 C.F.R. 1.821-1.825 (“Requirements for Patent Applications Containing Nucleotide Sequences and/or Amino Acid Sequence Disclosures—the Sequence Rules”) and are consistent with World Intellectual Property Organization (WIPO) Standard ST.25 (1998) and the sequence listing requirements of the EPC and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

SEQ ID NOs: 1-35 are the amino acid sequences of peptides having strong affinity for a surface comprising PMMA polymer.

SEQ ID NO: 5 is the amino acid sequence of a peptide classified herein as Group “A” PMMA polymer-binding peptides. Examples of PMMA-binding peptides belonging to Group “A” are selected from the group consisting of SEQ ID NOs: 1, 2, 3, and 4 and 5.

SEQ ID NO: 9 is the amino acid sequence of a peptide classified herein as Group “B” PMMA polymer-binding peptides. Examples of PMMA-binding peptides belonging to Group “B” are selected from the group consisting of SEQ ID NOs: 6, 7, 8 and 9.

SEQ ID NO: 14 is the amino acid sequence of a peptide classified herein as Group “C” PMMA polymer-binding peptides. Examples of PMMA-binding peptides belonging to Group “C” are selected from the group consisting of SEQ ID NOs: 10, 11, 12, 13, and 14.

SEQ ID NO: 18 is the amino acid sequence of a peptide classified herein as Group “D” PMMA polymer-binding peptides. Examples of PMMA-binding peptides belonging to Group “D” are selected from the group consisting of SEQ ID NOs: 15, 16, 17, and 18.

SEQ ID NO: 21 is the amino acid sequence of a peptide classified herein as Group “E” PMMA polymer-binding peptides. Examples of PMMA-binding peptides belonging to Group “E” are selected from the group consisting of SEQ ID NOs: 19, 20, and 21.

SEQ ID NO: 24 is the amino acid sequence of a peptide classified herein as Group “F” PMMA polymer-binding peptides. Examples of PMMA-binding peptides belonging to Group “F” are selected from the group consisting of SEQ ID NOs: 22, 23, and 24.

SEQ ID NO: 27 is the amino acid sequence of a peptide classified herein as Group “G” PMMA polymer-binding peptides. Examples of PMMA-binding peptides belonging to Group “G” are selected from the group consisting of SEQ ID NOs: 25, 26, and 27.

SEQ ID NOs: 28-35 are additional amino acid sequences of peptides having strong affinity for PMMA polymer.

SEQ ID NO: 36 is the amino acid sequence of the N-terminal constant region used in the present display library.

SEQ ID NO: 37 is the amino acid sequence of the C-terminal constant region used in the present display library.

SEQ ID NO: 38 is the nucleic acid sequence of the oligonucleotide portion of the MHA-oligonucleotide linker used in preparing the fusion molecules.

SEQ ID NOs: 39 and 40 are primers.

SEQ ID NO: 41 is the amino acid sequence of the Caspase-3 cleavage sequence.

SEQ ID NOs: 42-100 are the amino acid sequence of polymer-binding peptides.

SEQ ID NOs: 101-104 are the amino acid sequence of cellulose acetate-binding peptides.

SEQ ID NOs: 105-159 are the amino acid sequences of pigment-binding peptides.

SEQ ID NOs: 160-174 are the amino acid sequence of clay-binding peptides.

SEQ ID NOs: 175-200 are the amino acid sequences of calcium carbonate-binding peptides.

SEQ ID NOs: 201-223 are the amino acid sequences of silica-binding peptides.

SEQ ID NOs: 224-252 are the amino acid sequences of antimicrobial peptides.

SEQ ID NOs: 253-254 are the amino acid sequences of several peptide linkers.

SEQ ID NOs: 255-256 are the amino acid sequences of several peptide bridges.

SEQ ID NO: 257 is the amino acid sequence of PMMA-binding peptide of SEQ ID NO: 6 further comprising a C-terminal lysine residue.

SEQ ID NOs: 258-474 are examples of peptides having affinity for a body surface wherein SEQ ID NOs: 258-384 bind to hair; SEQ ID NOs 380-432 binding to skin; SEQ ID NOs: 433-434 bind to nail; SEQ ID NOs: 435-454 bind to tooth pellicle; and SEQ ID NOs: 455-474 bind to tooth enamel.

### DETAILED DESCRIPTION OF THE INVENTION

Provided herein are peptides having strong affinity for PMMA (PMMA-binding peptides) as well as peptide-based reagents comprising at least one of said PMMA-binding peptides. The peptide-based reagents are useful for coupling a benefit agent to a surface comprising PMMA polymer or for coupling at least one first surface comprising polymethyl methacrylate (PMMA) polymer to at least one second target surface. For example, a particulate benefit agent comprising a surface of PMMA polymer, such as a PMMA-coated pigment, can be coupled to a second surface, such as a body surface. The first and second target surface may be the same or different so long as at least one of the surfaces comprises a surface of PMMA polymer.

In this disclosure, a number of terms and abbreviations are used. The following definitions apply unless specifically stated otherwise.

As used herein, the articles “a”, “an”, and “the” preceding an element or component of the invention are intended to be nonrestrictive regarding the number of instances (i.e., occurrences) of the element or component. Therefore “a”, “an” and “the” should be read to include one or at least one, and the singular word form of the element or component also includes the plural unless the number is obviously meant to be singular.

The term “comprising” means the presence of the stated features, integers, steps, or components as referred to in the claims, but does not preclude the presence or addition of one or more other features, integers, steps, components or groups thereof. The term “comprising” is intended to include embodiments encompassed by the terms “consisting essentially of” and “consisting of”. Similarly, the term “consisting essentially of” is intended to include embodiments encompassed by the term “consisting of”.

As used herein, the term “about” modifying the quantity of an ingredient or reactant employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities.

Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, and the like.

As used herein, the terms “polypeptide” and “peptide” will be used interchangeably to refer to a polymer of two or more amino acids joined together by a peptide bond. In one aspect, this term also includes post expression modifications of the polypeptide, for example, glycosylations, acetylations, phosphorylations and the like. Included within the definition are, for example, peptides containing one or more analogues of an amino acid or labeled amino acids and peptidomimetics. In one embodiment, the peptides are comprised of L-amino acids.

As used herein, the term “polymethyl methacrylate” polymer is abbreviated as “PMMA” and is synonymous with methyl 2-methylpropanoate, poly (methyl methacrylate) and all other synonyms used under CAS #9011-14-7.

As used herein, “PBP” means a PMMA-binding peptide. As used herein, the term “PMMA-binding peptide” refers to peptides that bind with strong affinity to the surface of a PMMA polymer or copolymer comprising PMMA. In one embodiment, the PMMA-binding peptide binds to PMMA polymer. For purposes of the present discussion PMMA-binding peptides have classed into various Groups ranging from Group A to H, based on specific differentiating amino acid motifs within each group. The present application relates to Group A PMMA-binding peptides.

As used herein, the term “peptide finger” will be used to refer to an individual target surface-binding peptide, typically identified by biopanning against a target surface. Peptides having affinity for PMMA by biopanning may be referred to as “PMMA-binding peptides” or peptide “fingers”.

As used herein, the term “peptide hand” will be used to refer to a binding domain or region comprising 2 or more “peptide fingers” coupled together using one or more optional, independently-selected linkers, wherein the inclusion of at least one peptide linker is preferred.

As used herein, the terms “PMMA hand” and “PMMA-binding domain” will refer to a single chain peptide comprising of at least two PMMA-binding peptides linked together by an optional molecular linker (L) (“linker”) or spacer, wherein the inclusion of a molecular linker is preferred. In one embodiment, the molecular linker is a peptide linker. In another embodiment, the peptide linker ranges in length from 1 to 50 amino acids, preferably 3 to 25 amino acids in length, and may be comprised of various amino acids. In another embodiment, the molecular linker may be comprised of one or more of the amino acids selected from the group consisting of proline, lysine, glycine, alanine, glutamic acid, serine, and combinations thereof.

As used herein, the term “peptide-based reagent” or “peptide reagent” refers to a single chain peptide comprising at least one of the present PMMA-binding peptides having an amino acid sequence (SEQ ID NO: 14). In one embodiment, the peptide reagent comprises at least one of the present PMMA-binding peptides having an amino acid sequence selected from the group consisting of SEQ ID NOs: 10, 11, 12, 13, and 14. In another embodiment, the peptide reagent comprises at least one PMMA-binding peptides having an amino acid sequence selected from the group consisting of SEQ ID NOs: 10, 11, 12, and 13.

In another embodiment, the peptide-based reagent comprises two or more of the present PMMA-binding peptides separated by a molecular linker. The peptide-based reagent may also have at least one region that can be coupled to the benefit agent and/or a region that provides a binding affinity for a second target surface. As such, the peptide-based reagent may used as an interfacial material to couple a benefit agent or an additional target surface (via a target surface-binding domain or “TBD”) to a surface comprised of PMMA polymer. The benefit agent-binding region may be comprised of at least benefit agent-binding peptide. The benefit agent may be coupled covalently or non-covalently to the present peptide-based reagents. In one embodiment, the benefit agent is couple non-covalently to the peptide reagent. In another embodiment, the benefit agent is coupled to the peptide-based reagent covalently.

As used herein, the terms “coupling” and “coupled” refer to any chemical association and may include both covalent and non-covalent interactions. In one embodiment, the coupling is non-covalent. In another embodiment, the coupling is covalent.

As used herein, the term “bridge”, “peptide bridge”, and “bridging element” will refer to a linear peptide used to join a PMMA-binding domain (“PMMA-binding hand” or the “first domain”) to a peptide domain (the “second domain”) capable of binding to the surface of particulate benefit agent (i.e., covalent or non-covalent coupling) or a second target surface via a target surface-binding domain (TBD). The peptide bridge may range in size from 1 to 60 amino acids in length, preferably 6 to 30 amino acids in length. Examples of peptide bridges are provided as SEQ ID NOs: 255-256.

The term “benefit agent” is abbreviated as “BA” and is a general term applying to a compound or substance that may be coupled to a surface comprising PMMA polymer using one of the present PMMA-binding peptides or peptide-based reagents in order to provide a desirable characteristic of the benefit agent to the complex. In the most general sense a benefit agent may be any element, molecule or compound that is not PMMA. In one embodiment, the benefit agent may be one or more of the PMMA-binding peptides. Benefit agents typically include, but are not limited to, colorants such as pigments and dyes as well as pharmaceuticals, markers, conditioners, fragrances, as well as domains having a defined activity (“active domains” or “AD”) such as enzyme catalysts, and antimicrobial agents, such as antimicrobial peptides.

The term “target binding domain” is abbreviated as “TBD” will refer to a portion or region of the peptide-based reagent having affinity for a target surface. In one embodiment, the TBD has strong affinity for a target surface. In another embodiment, the present peptide-based reagents will comprise at least one region or domain having strong affinity for a surface comprising PMMA, wherein the domain having affinity for PMMA will be comprises of at least one of the present PMMA-binding peptides; and at least one second region or domain having strong affinity for a benefit agent or another target surface including, but not limited to, body surfaces such as hair, skin, nails, teeth, gums, and corneal tissue, as well as other surfaces such as pigments, synthetic polymers, peptides, nucleic acids, conditioning agents, print media, clay, calcium carbonate, silica, and other particulate benefit agents, such as microspheres. In one embodiment, the target binding domain (TBD) is a body surface-binding domain selected from the group consisting of a hair-binding domain, a skin-binding domain, a nail-binding domain, a tooth-binding domain (both tooth pellicle-binding peptides and/or tooth enamel-binding peptides), and domains having affinity for other body surfaces, such as the gums or corneal tissue. Examples of various peptides having affinity various benefit agent surfaces are provided in the present sequence descriptions and the accompanying sequence listing.

The term “body surface” will mean any surface of the human body that may serve as a substrate for the binding of a peptide carrying a benefit agent. Typical body surfaces may include, but are not limited to, hair, skin, nails, teeth (enamel and/or pellicle surfaces), gums, and corneal tissue. In one embodiment, the body surface is selected from the group consisting of hair, skin, nail, tooth enamel, and tooth pellicle.

As used herein, “BSBP” means body surface-binding peptide. A body surface-binding peptide is a peptide having strong affinity for a specified body surface. A body surface-binding peptide is a peptide ranging in size from 7 to 60 amino acids in length that binds with strong affinity to at least one body surface. As used herein, the body surface-binding peptide is selected from the group consisting of hair-binding peptides, skin-binding peptides, nail-binding peptides, and oral cavity surface-binding peptides, such as a tooth enamel-binding peptides and tooth pellicle-binding peptides. In a preferred embodiment, the body surface-binding peptide is selected from the group consisting of a hair-binding peptide, a skin-binding peptide, a nail-binding peptide, and a tooth-binding peptide (enamel or pellicle). Examples of body surface-binding peptides are provided as SEQ ID NOs: 258-474.

As used herein, the term “hair” as used herein refers to human hair, eyebrows, and eyelashes. The term “hair surface” will mean the surface of human hair capable of binding to a hair-binding peptide. As used herein, the term “hair-binding peptide” refers to a peptide that binds with high affinity to hair. Examples of hair-binding peptides are described in U.S. Patent Application Publication NOs. 2005-0226839; 2007-0065387; 2007-0110686; 2007-0196305; U.S. patent application Ser. Nos. 11/877,692 and 11/939,583; U.S. Pat. No. 7,220,405; and published PCT Application No. WO2004/048399. Examples of hair-binding peptides are provided as SEQ ID NOs: 258-384.

The term “skin”, as used herein, refers to human skin, or pig skin, VITRO-SKIN® and EPIDERM™ which are substitutes for human skin. Skin will generally comprise a layer of epithelial cells and may additionally comprise a layer of endothelial cells. Examples of skin-binding peptides are described in U.S. Patent Application Publication NOs. 2005-0249682; US 2006-0199206; 2007-0065387; and 2007-0110686; U.S. patent application Ser. No. 11/877,692; and published PCT Application NO. WO2004/048399.

As used herein, the term “skin-binding peptide” refers to peptides that bind with strong affinity to skin. Examples of skin-binding peptides have also been reported (U.S. Patent Application Publication NOs. 2007-0274931 and 2007-0249805, and published PCT Patent Application WO 2004/000257). The skin-binding peptides may be linked together to form skin-binding domains (“hands”). Examples of skin-binding peptides are provided as SEQ ID NOs: 380-432.

As used herein, the term “nails” as used herein refers to human fingernails and toenails. As used herein, the term “nail-binding peptide” refers to peptides that bind with strong affinity to nails. Examples of nail-binding peptides are provided as SEQ ID NOs: 433-434. The nail-binding fingers may be linked together to form nail-binding domains (“hands”).

As used herein, the term “oral cavity surface-binding peptide” refers to peptides that bind with strong affinity to teeth, gums, cheeks, tongue, or other surfaces in the oral cavity. As used herein, the term “tooth-binding peptide” will refer to a peptide that binds with high affinity to tooth enamel or tooth pellicle. Examples of tooth-binding peptides are disclosed in co-pending U.S. Patent Application Publication NO. 2008-0280810 and are provided as SEQ ID NOs: 435-474. The tooth-binding fingers may be linked together to form tooth-binding domains (“hands”). In one embodiment, the oral cavity surface-binding peptide is a peptide that binds with high affinity to a tooth surface.

The term “tooth surface” will refer to a surface comprised of tooth enamel (typically exposed after professional cleaning or polishing) or tooth pellicle (an acquired surface comprising salivary proteins). Hydroxyapatite can be coated with salivary glycoproteins to mimic a natural tooth pellicle surface (tooth enamel is predominantly comprised of hydroxyapatite).

As used herein, the terms “pellicle” and “tooth pellicle” will refer to the thin film (typically ranging from about 1 μm to about 200 μm thick) derived from salivary glycoproteins which forms over the surface of the tooth crown. Daily tooth brushing tends to only remove a portion of the pellicle surface while abrasive tooth cleaning and/or polishing (typically by a dental professional) will exposure more of the tooth enamel surface.

As used herein, the terms “enamel” and “tooth enamel” will refer to the highly mineralized tissue which forms the outer layer of the tooth. The enamel layer is composed primarily of crystalline calcium phosphate (i.e., hydroxyapatite) along with water and some organic material. In one embodiment, the tooth surface is selected from the group consisting of tooth enamel and tooth pellicle.

As used herein, the term “pigment” means an insoluble colorant. A wide variety of organic and inorganic pigments alone or in combination may be used. In one embodiment, the pigment is a metal oxide. As used herein, the term “pigment lake” or “lake” refers to a pigment manufactured by precipitating a dye with an inert binder, usually a metallic salt.

As used herein, “Pigment-BP” means pigment-binding peptide. A pigment-binding peptide is a peptide that binds with strong affinity to a specified pigment. Pigment-binding peptides have been reported in the art (U.S. Patent Application Publ. No. 2005-0054752, U.S. Pat. No. 7,285,264, and co-pending U.S. patent application Ser. No. 12/632,827). Examples of pigment-binding peptides are provided as SEQ ID NOs: 105-159. Examples of iron oxide-based pigment binding peptides are provided as SEQ ID NOs: 131-159 (U.S. patent application Ser. No. 12/632,827).

As used herein, a “polymer” is a natural or synthetic compound of usually high molecular weight consisting of repeated linked units. As used herein, “Poly-BP” means polymer-binding peptide (excluding the PMMA-binding peptides (SEQ ID NOs: 1-35)). Examples of peptides that bind with high affinity to a specified polymer have been described (U.S. Patent Application Publication No. 2008-0206809). Examples of polymer-binding peptides may include peptides that bind to (previously reported) polymethyl methacrylate (SEQ ID NOs: 42-68), polypropylene (SEQ ID NOs: 69-75), polytetrafluoroethylene (SEQ ID NOs: 76-84), polyethylene (85-91), nylon (SEQ ID NOs: 92-97), and polystyrene (SEQ ID NOs: 98-100).

Additional peptides having strong affinity for their respective surfaces also include, but are not limited to, cellulose acetate-binding peptides (SEQ ID NOs: 101-104); silica-binding peptides (U.S. patent application Ser. No. 12/632,829 and SEQ ID NOs: 201-223); clay-binding peptides (U.S. Patent Application Publication No. 2007-0249805 and SEQ ID NOs: 160-174); and calcium carbonate-binding peptides (U.S. Patent Application Publication No. 2009-0029902 and SEQ ID NOs: 175-200).

As used herein, an “antimicrobial peptide” is a peptide having the ability to kill microbial cell populations (see U.S. Pat. No. 7,427,656). Examples of antimicrobial peptides are provided as SEQ ID NOs: 224-252.

As used herein, the term “operably-linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). In a further embodiment, the definition of “operably linked” may also be extended to describe the products of chimeric genes.

As used herein, the term “MB₅₀” refers to the concentration of the binding peptide that gives a signal that is 50% of the maximum signal obtained in an ELISA-based binding assay (see Example 9 of U.S. Published Patent Application No. 2005-0226839; hereby incorporated by reference). The MB₅₀ provides an indication of the strength of the binding interaction or affinity of the components of the complex. The lower the value of MB₅₀, the stronger the interaction of the peptide with its corresponding substrate.

As used herein, the terms “binding affinity” and “affinity” refer to the strength of the interaction of a binding peptide (e.g., target surface-binding peptides, target surface-binding domains, and peptide-based reagents) with its respective substrate. The binding affinity may be reported in terms of the MB₅₀ value as determined in an ELISA-based binding assay or as a K_{D} (equilibrium dissociation constant) value, which may be deduced using a methodology such as surface plasmon resonance (SPR).

As used herein, the term “strong affinity” refers to a binding affinity, as measured as an MB₅₀ value of K_{D} value, of 10⁻⁴ M or less, preferably less than 10⁻⁵ M, more preferably less than 10⁻⁶ M, more preferably less than 10⁻⁷ M, even more preferably less than 10⁻⁸ M, and most preferably less than 10⁻⁹ M.

As used herein, “L” means “molecular linker” or “linker”. The linker may be a peptide or non-peptide-based molecular linker. In one embodiment, the linker is a peptide linker. Peptide linkers separating a PMMA-binding domain from a benefit agent, a benefit agent-binding domain or a target surface-binding domain (TBD) may also be referred to as a peptide “bridge” or “bridging element”. In one embodiment, the peptide linker is 1 to 60 amino acids in length, preferably 3 to 25 amino acids in length. Examples of peptide linkers are provided as SEQ ID NOs: 253-254.

In one embodiment, the benefit agent may be an active domain within (i.e., a subsequence of the peptide reagent) or coupled to the peptide reagent. In one embodiment, the active domain is a portion of the peptide reagent that is not responsible for PMMA binding but provides additional functionality or benefit. In another embodiment the active domain may have antimicrobial functionality. For example, the peptide reagent may be comprised of at least one of the present PMMA-binding peptides and at least one antimicrobial peptide; whereby coupling of said peptide reagent to a surface comprising PMMA polymer (such as PLEXIGLAS™) provides a surface characterized by an enhancement in antimicrobial activity.

The term “amino acid” refers to the basic chemical structural unit of a protein or polypeptide. The following abbreviations are used herein to identify specific amino acids:

| | Three-Letter | One-Letter |
|---|---|---|
| Amino Acid | Abbreviation | Abbreviation |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Miscellaneous (or as defined herein) | Xaa | X |

### Binding Affinity

The present PMMA-binding peptides exhibit a strong affinity for a surface comprising PMMA polymer based on their ability to bind to a PMMA polymer after many rounds of selection under stringent selection conditions. The affinity of the peptide for PMMA can be expressed in terms of the dissociation constant K_{D} or an ELISA-based MB₅₀ value. K_{D} (expressed as molar concentration) corresponds to the concentration of peptide at which the binding site on the target is half occupied, i.e. when the concentration of target with peptide bound (bound target material) equals the concentration of target with no peptide bound. The smaller the dissociation constant, the more tightly bound the peptide is; for example, a peptide with a nanomolar (nM) dissociation constant binds more tightly than a peptide with a micromolar (μM) dissociation constant. In one embodiment, the present PMMA-binding peptides have a K_{D} value of 10⁻⁴ M or less, preferably 10⁻⁵ M or less, more preferably 10⁻⁶ M or less, even more preferably 10⁻⁷ M or less, yet even more preferably 10⁻⁸ M or less, and most preferably 10⁻⁹ M or less.

Alternatively, one of skill in the art can also use an ELISA-based assay to calculate a relative affinity of the peptide for the target material (reported as an MB₅₀ value; see present Example 3 and co-owned U.S. Patent Application Publication No. 2005-022683, herein incorporated by reference). As used herein, the term “MB₅₀” refers to the concentration of the binding peptide that gives a signal that is 50% of the maximum signal obtained in an ELISA-based binding assay. The MB₅₀ value provides an indication of the strength of the binding interaction or affinity of the components of the complex. The lower the value of MB₅₀, the stronger the interaction of the peptide with its corresponding substrate. In one embodiment, the MB₅₀ value (reported in terms of molar concentration) for the PMMA-binding peptide is 10⁻⁴ M or less, preferably 10⁻⁵ M or less, more preferably 10⁻⁶ M or less, more preferably 10⁻⁷ M or less, and most preferably 10⁻⁸ M or less.

mRNA-Display

The present PMMA-binding peptides were biopanned against a PMMA polymer using mRNA display, an in vitro panning method commonly used for identifying peptides having an affinity for a target material (U.S. Pat. No. 6,258,558). Briefly, a random library of DNA molecules was generated wherein they encode a peptide of a desired length. The length of the peptide within the display library may be to be up to 200 amino acids in length and is typically designed to range from about 7 to about 100 amino acids in length. In one embodiment, the library of peptides may be designed to be about 7 to about 60 amino acids in length, preferably about 7 to about 30 amino acids in length, more preferably about 15 to about 30 amino acids in length, and most preferably about 27 amino acids in length (i.e., a “27-mer” library). Typically, the nucleic acid molecule encoding the peptide includes (in addition to the coding region) appropriate 5′ and 3′ regulatory regions necessary for efficient in vitro transcription and translation. The design of the nucleic acid constructs used for preparing the mRNA-display library is well known to one of skill in the (see WO2005/051985). The nucleic acid molecules can be designed to optionally encode flexible linkers, cleavage sequences, fusion promoting sequences, and identification/purification tags (e.g., poly-A regions, His tags, etc.) to facility purification and/or processing in subsequence steps.

The library of random nucleic acid fragments is transcribed in vitro to produce an mRNA library. The mRNA is isolated and subsequently fused to a linker molecule (i.e., a puromycin-oligonucleotide linker or a puromycin derivative-oligonucleotide linker) using techniques well-known in the art (U.S. Pat. No. 6,258,558; U.S. Pat. No. 6,228,994; and Kurz et al., (2000) NAR, 28(18):e83 i-v). In a preferred embodiment, the puromycin-oligonucleotide linker comprises psoralen for rapid and facile preparation of the mRNA-protein fusions (Kurtz et al., supra). The mRNA-puromycin fusion molecules are then translated in vitro whereby the nascent polypeptide is fused (via the puromycin-oligonucleotide linker) to the mRNA (PROFUSION™ molecules; Adnexus Therapeutics, Weltham, Mass.). In this way, the phenotype (peptide) is linked to the corresponding genotype (RNA).

The mRNA-peptide fusion molecules are typically reverse transcribed into a DNA/mRNA-protein fusion molecules prior to affinity selection. The library (often comprising up to 10¹³ different sequences) is contacted with target ligand/material (typically an immobilized target and/or a solid surface). The selection process is carried out in an aqueous medium wherein parameters such as time, temperature, pH, buffer, salt concentration, and detergent concentration may be varied according the stringency of the selection strategy employed. Typically, the temperature of the incubation period ranges from 0° C. to about 40° C. and the incubation time ranges from about 1 to about 24 hours.

Several washing steps are typically used to remove the non-binding/low affinity fusion molecules. The stringency of the washing conditions may be adjusted to select those fusion molecules having the highest affinity for the target material. The high affinity fusion molecules are isolated and then PCR-amplified in order to obtain the nucleic acid sequences encoding the binding peptides. The mRNA-display selection cycle is typically repeated for 3 to 10 cycles in order to select/enrich those fusion molecules comprising peptide sequences exhibiting the highest affinity for the target material.

Error-prone PCR may optionally be incorporated into mRNA-display selection process whereby mutants derived from a previously selected high affinity sequence are used. The process is typically repeated for several cycles in order to obtain the peptides having improved affinity for the target material.

Optionally, any PMMA-binding peptide sequence identified using mRNA-display may be verified using the free peptide. Typically, the nucleic acid molecule encoding the PMMA-binding peptide is cloned and recombinantly expressed in an appropriate microbial host cell, such as E. coli. The free peptide is then isolated and assayed against the targeted material to validate the binding affinity of the peptide sequence.

### Polymethyl Methacrylate

PMMA polymer is prepared by the polymerization of the monomer methyl methacrylate, which is available from many commercial suppliers, such as Aldrich (Milwaukee, Wis.), ICI Acrylics (Beaumont, Tex.), CYRO Industries (Rockaway, N.J.), Total Specialty Chemicals, Inc (New Canaan, Conn.), and Degussa Corp. (Parsippani, N.J.). Methyl methacrylate may be polymerized using methods known in the art, such as radical polymerization, anionic polymerization, or group transfer polymerization (Ullmann's Encyclopedia of Industrial Chemistry, 6^{th} edition, 2003, Wiley-VCH Verlag GmbH and Co., Weinheim, Germany, Vol. 28, pp. 377-389). For example, radical polymerization may be carried out homogeneously (i.e., bulk or solution polymerization) or heterogeneously (i.e., suspension or emulsion polymerization). The radical polymerization may be initiated using radiation, heat, or chemical initiators, such as azo compounds or organic peroxy compounds. Copolymers may be produced by these methods using a mixture of the desired monomers.

The PMMA polymer may be produced in various shapes or forms, such as beads, microspheres, sheets, rods, tubes, films, plates, rings, fiber, and microfilament, using injection molding, extrusion, and casting techniques, which are well known in the art. Additionally, PMMA in various shapes is available commercially from companies such as CRYO Industries and Bang Laboratories (Fishers, Ind.).

In one embodiment, the PMMA polymer or a copolymer prepared using PMMA is coated onto another surface, such as metal, metal oxide, polymer, pigment, glass, cloth, and the like, using methods known in the art, such as spraying, brushing, dip coating and casting.

In another embodiment, the PMMA polymer or copolymer is imbedded into the surface of another material, such as another polymer. This may be done by adding particles, beads, or fragments of PMMA material into the other polymer as it cures.

In another embodiment, a PMMA copolymer is used as a dispersant for pigments or other insoluble particles, including metallic and semiconductor nanoparticles. The copolymer may be a random copolymer or a structured copolymer (i.e., a non-random block copolymer). Preferred random dispersants may include methyl methacrylate copolymers with other acrylates or styrene. Most preferred are structured polymer dispersants, which include AB, BAB and ABC block copolymers, branched polymers and graft polymers. Preferably these copolymers comprise methyl methacrylate with one or more monomers such as acrylate, methacrylate, butyl methacrylate, 2-ethylhexyl methacrylate, benzylmethacrylate, phenoxyethyl acrylate, and ethoxytriethyleneglycolmethacrylate, such as those described by Nigan (U.S. Patent Application Publication No. 2004-0232377). Some useful structured polymer dispersants are disclosed in U.S. Pat. No. 5,085,698, EP-A-0556649, and U.S. Pat. No. 5,231,131.

### Production of Peptides

The present peptides may be prepared using standard peptide synthesis methods, which are well known in the art (see for example Stewart et al., Solid Phase Peptide Synthesis, Pierce Chemical Co., Rockford, Ill., 1984; Bodanszky, Principles of Peptide Synthesis, Springer-Verlag, New York, 1984; and Pennington et al., Peptide Synthesis Protocols, Humana Press, Totowa, N.J., 1994). Additionally, many companies offer custom peptide synthesis services.

Alternatively, the present peptides may be prepared using recombinant DNA and molecular cloning techniques. Genes encoding the present peptides may be produced in heterologous host cells, particularly in the cells of microbial hosts, as described by Huang et al. (U.S. Patent Application Publication No. 2005-0050656).

Preferred heterologous host cells for expression of the present peptides are microbial hosts that can be found broadly within the fungal or bacterial families and which grow over a wide range of temperature, pH values, and solvent tolerances. Because transcription, translation, and the protein biosynthetic apparatus are the same irrespective of the cellular feedstock, functional genes are expressed irrespective of carbon feedstock used to generate cellular biomass. Examples of host strains include, but are not limited to, bacterial, fungal or yeast species such as Aspergillus, Trichoderma, Saccharomyces, Pichia, Phaffia, Kluyveromyces, Candida, Hansenula, Yarrowia, Salmonella, Bacillus, Acinetobacter, Zymomonas, Agrobacterium, Erythrobacter, Chlorobium, Chromatium, Flavobacterium, Cytophaga, Rhodobacter, Rhodococcus, Streptomyces, Brevibacterium, Corynebacteria, Mycobacterium, Deinococcus, Escherichia, Erwinia, Pantoea, Pseudomonas, Sphingomonas, Methylomonas, Methylobacter, Methylococcus, Methylosinus, Methylomicrobium, Methylocystis, Alcaligenes, Synechocystis, Synechococcus, Anabaena, Thiobacillus, Methanobacterium, Klebsiella, and Myxococcus. In one embodiment, bacterial host strains include Escherichia, Bacillus, and Pseudomonas. In a preferred embodiment, the bacterial host cell is Escherichia coli.

### Benefit Agents

Benefit agents are any material or substance that may be complexed with the peptide-based reagent comprising one or more of the present PMMA-binding peptides in an manner so as to deliver a benefit at the point where the peptide reagent is attached. A benefit agent may be selected for the purpose of adding the physical, chemical and/or biological properties of said agent to the PMMA polymer surface.

Benefit agents may be inorganic or organic in nature. Some preferred embodiments include benefit agents that are pigments, conditioners, colorants, antimicrobial agents, and fragrances.

### Conditioners

In one embodiment, a peptide-based reagent may be used that provides a conditioning effect to a body surface. For example, a peptide reagent may be designed to couple a target surface, such as a body surface, with a conditioning agent comprising a surface of PMMA polymer. The conditioning agent may be provided or incorporated with a bead, particle, or microsphere comprising a PMMA polymer or copolymer surface. Conditioner benefits agents as referred to in discussion of the present invention generally mean benefit agents that provide an improvement to the appearance, texture or quality of the substance they are designed to condition. Conditioner benefit agents may be used with the present invention to condition any substance including but not limited to hair, skin, nail, tooth enamel, tooth pellicle, gums, others tissues of the oral cavity, leather, and upholstery. In the preferred embodiment the present invention is used in combination with a benefit agent that provides a conditioning effect to hair, skin, nails, tooth enamel, and tooth pellicle.

Hair conditioning agents are well known in the art, see for example Green et al. (WO 01/07009) and are available commercially from various sources. Suitable examples of hair conditioning agents include, but are not limited to cationic polymers, such as cationized guar gum, diallyl quaternary ammonium salt/acrylamide copolymers, quaternized polyvinylpyrrolidone and derivatives thereof, and various polyquaternium-compounds; cationic surfactants, such as stearalkonium chloride, centrimonium chloride, and Sapamin hydrochloride; fatty alcohols, such as behenyl alcohol; fatty amines, such as stearyl amine; waxes; esters; nonionic polymers, such as polyvinylpyrrolidone, polyvinyl alcohol, and polyethylene glycol; silicones; siloxanes, such as decamethylcyclopentasiloxane; polymer emulsions, such as amodimethicone; and volumizing agents, such as nanoparticles (e.g., silica nanoparticles and polymer nanoparticles). The preferred hair conditioning agents contain amine or hydroxyl functional groups to facilitate coupling to the hair-binding peptides. Examples of conditioning agents are octylamine (CAS No. 111-86-4), stearyl amine (CAS No. 124-30-1), behenyl alcohol (CAS No. 661-19-8, Cognis Corp., Cincinnati, Ohio), vinyl group terminated siloxanes, vinyl group terminated silicone (CAS No. 68083-19-2), vinyl group terminated methyl vinyl siloxanes, vinyl group terminated methyl vinyl silicone (CAS No. 68951-99-5), hydroxyl terminated siloxanes, hydroxyl terminated silicone (CAS No. 80801-30-5), amino-modified silicone derivatives, [(aminoethyl)amino]propyl hydroxyl dimethyl siloxanes, [(aminoethyl)amino]propyl hydroxyl dimethyl silicones, and alpha-tridecyl-omega-hydroxy-poly(oxy-1,2-ethanediyl) (CAS No. 24938-91-8).

If the present peptide-based reagents are to be used in connection with a hair care composition, such as when the target binding domain (TBD) of the peptide reagent has affinity for hair, an effective amount of the peptide reagent (alone or in a complex with a PMMA-coated benefit agent) for use in a hair care composition is herein defined as a proportion of from about 0.01% to about 10%, preferably about 0.01% to about 5% by weight relative to the total weight of the composition. Components of a cosmetically acceptable medium for hair care compositions are described by Philippe et al. in U.S. Pat. No. 6,280,747, and by Omura et al. in U.S. Pat. No. 6,139,851 and Cannell et al. in U.S. Pat. No. 6,013,250, each of which is incorporated herein by reference. For example, these hair care compositions can be aqueous, alcoholic or aqueous-alcoholic solutions, the alcohol preferably being ethanol or isopropanol, in a proportion of from about 1 to about 75% by weight relative to the total weight, for the aqueous-alcoholic solutions. Additionally, the hare care compositions may contain one or more conventional cosmetic or dermatological additives or adjuvants including but not limited to, antioxidants, preserving agents, fillers, surfactants, UVA and/or UVB sunscreens, fragrances, thickeners, wetting agents and anionic, nonionic or amphoteric polymers, and dyes or pigments.

Skin conditioning agents may include, but are not limited to, astringents, which tighten skin; exfoliants, which remove dead skin cells; emollients, which help maintain a smooth, soft, pliable appearance; humectants, which increase the water content of the top layer of skin; occlusives, which retard evaporation of water from the skin's surface; and miscellaneous compounds that enhance the appearance of dry or damaged skin or reduce flaking and restore suppleness. Particles comprising PMMA and a skin conditioning agent may be in conjunction with one of the present peptide-based reagents to couple the condition agent to skin (assuming the peptide reagent also comprises a portion having affinity for skin). Skin conditioning agents are well known in the art, see for example Green et al., supra, and are available commercially from various sources. Suitable examples of skin conditioning agents include, but are not limited to alpha-hydroxy acids, beta-hydroxy acids, polyols, hyaluronic acid, D,L-panthenol, polysalicylates, vitamin A palmitate, vitamin E acetate, glycerin, sorbitol, silicones, silicone derivatives, lanolin, natural oils and triglyceride esters. The skin conditioning agents may also include polysalicylates, propylene glycol (CAS No. 57-55-6, Dow Chemical, Midland, Mich.), glycerin (CAS No. 56-81-5, Proctor & Gamble Co., Cincinnati, Ohio), glycolic acid (CAS No. 79-14-1, DuPont Co., Wilmington, Del.), lactic acid (CAS No. 50-21-5, Alfa Aesar, Ward Hill, Mass.), malic acid (CAS No. 617-48-1, Alfa Aesar), citric acid (CAS No. 77-92-9, Alfa Aesar), tartaric acid (CAS NO. 133-37-9, Alfa Aesar), glucaric acid (CAS No. 87-73-0), galactaric acid (CAS No. 526-99-8), 3-hydroxyvaleric acid (CAS No. 10237-77-1), salicylic acid (CAS No. 69-72-7, Alfa Aesar), and 1,3 propanediol (CAS No. 504-63-2, DuPont Co., Wilmington, Del.). Polysalicylates may be prepared by the method described by White et al. in U.S. Pat. No. 4,855,483, incorporated herein by reference. Glucaric acid may be synthesized using the method described by Merbouh et al. (Carbohydr. Res., (2001) 336:75-78). The 3-hydroxyvaleric acid may be prepared as described by Bramucci et al. in U.S. Pat. No. 6,562,603.

In a number of embodiments the present peptide reagents could be used in a skin care composition (for example, when the peptide reagent comprises a skin-binding domain and a PMMA polymer binding domain, wherein the benefit agent comprise PMMA polymer, such as a bead or surface coating). Skin care compositions are herein defined as compositions comprising an effective amount of a skin conditioner or a mixture of different skin conditioners in a cosmetically acceptable medium. The uses of these compositions include, but are not limited to, skin care, skin cleansing, make-up, and anti-wrinkle products. If the present invention is desired to be used in connection with a skin care composition an effective amount of the complex for skin care compositions is herein defined as a proportion of from about 0.001% to about 10%, preferably about 0.01% to about 5% by weight relative to the total weight of the composition. This proportion may vary as a function of the type of skin care composition. Suitable compositions for a cosmetically acceptable medium are described by Philippe et al., supra. For example, the cosmetically acceptable medium may be an anhydrous composition containing a fatty substance in a proportion generally of from about 10 to about 90% by weight relative to the total weight of the composition, where the fatty phase containing at least one liquid, solid or semi-solid fatty substance. The fatty substance includes, but is not limited to, oils, waxes, gums, and so-called pasty fatty substances. Alternatively, the compositions may be in the form of a stable dispersion such as a water-in-oil or oil-in-water emulsion. Additionally, the compositions may contain one or more conventional cosmetic or dermatological additives or adjuvants, including but not limited to, antioxidants, preserving agents, fillers, surfactants, UVA and/or UVB sunscreens, fragrances, thickeners, wetting agents and anionic, nonionic or amphoteric polymers, and dyes or pigments.

### Colorants.

The term colorant generally refers to a coloring agent. Colorants may be chemically organic or inorganic and may include pigments, lakes or dyes. The colorants may be prepared by covalently attaching at least one of the present PMMA-binding peptides to a coloring agent, either directly or via a linker, using any of the coupling methods known in the art (see for example, U.S. Patent Application Publication No. 2005-0226839).

Pigments are a particularly suitable benefit agent. A wide variety of organic and inorganic pigments alone or in combination may be used. Preferred organic pigments are carbon black, such as Carbon Black FW18, and colored pigments such as CROMOPHTAL® Yellow 131AK (Ciba Specialty Chemicals), SUNFAST® Magenta 122 (Sun Chemical) and SUNFAST® Blue 15:3 (Sun Chemical). Examples of inorganic pigments may include, but are not limited to finely divided metals such as copper, iron, aluminum, and alloys thereof; and metal oxides, such as silica, alumina, and titania. Additional examples of suitable pigments are given by Ma et al. in U.S. Pat. No. 5,085,698, incorporated herein by reference.

Suitable coloring agents that may be used with the present PMMA-binding peptides and/or peptide-based reagents may include, but are not limited to 4-hydroxypropylamino-3-nitrophenol, 4-amino-3-nitrophenol, 2-amino-6-chloro-4-nitrophenol, 2-nitro-paraphenylenediamine, N,N-hydroxyethyl-2-nitro-phenylenediamine, 4-nitro-indole, Henna, HC Blue 1, HC Blue 2, HC Yellow 4, HC Red 3, HC Red 5, Disperse Violet 4, Disperse Black 9, HC Blue 7, HC Blue 12, HC Yellow 2, HC Yellow 6, HC Yellow 8, HC Yellow 12, HC Brown 2, D&C Yellow 1, D&C Yellow 3, D&C Blue 1, Disperse Blue 3, Disperse violet 1, eosin derivatives such as D&C Red No. 21 and halogenated fluorescein derivatives such as D&C Red No. 27, D&C Red Orange No. 5 in combination with D&C Red No. 21 and D&C Orange No. 10; and pigments, such as D&C Red No. 36 and D&C Orange No. 17, the calcium lakes of D&C Red Nos. 7, 11, 31 and 34, the barium lake of D&C Red No. 12, the strontium lake of D&C Red No. 13, the aluminum lakes of FD&C Yellow No. 5, of FD&C Yellow No. 6, of D&C Red No. 27, of D&C Red No. 21, and of FD&C Blue No. 1, iron oxides, manganese violet, chromium oxide, titanium dioxide, zinc oxide, barium oxide, ultramarine blue, bismuth citrate, and carbon black particles.

### Fragrances.

The PMMA-binding peptides and/or peptide-based reagents may be used to delivery or couple a fragrance to a surface comprising PMMA. In another embodiment, a particle, bead, or microsphere comprising PMMA may also be used to delivery a fragrance to a target surface, such as a body surface, provided that the peptide reagent comprises an appropriate target binding domain (TBD), such as a body surface-binding domain.

A fragrance is a complex, compound or element that releases, a substance which may be perceived by the sense of olfaction or chemical detection in any organism, but preferably, in humans. The object sensed or detected may be a part of or the whole of the fragrance benefit agent. In the preferred embodiment the odor is perceived as desirable to humans. However, some uses may combine with a fragrance benefit agent that is repellent to a class of organisms, including a class that contains or is humans. Any known fragrance or odor may be use as a benefit agent. It may be desirable to attach a fragrance benefit agent to the PMMA-peptide complex by a bond structure or linking molecule that allows the benefit agent to be released, in part or in whole, so that it may be perceived by a sensing organ or chemical detector.

Numerous fragrances, both natural and synthetic, are well known in the art. For example, Secondini (Handbook of Perfumes and Flavors, Chemical Publishing Co., Inc., New York, 1990) describes many of the natural and synthetic fragrances used in cosmetics. Suitable natural fragrances may include, but are not limited to jasmine, narcissus, rose, violet, lavender, mint, spice, vanilla, anise, amber, orange, pine, lemon, wintergreen, rosemary, basil, and spruce. Suitable synthetic fragrances may include, but are no limited to, acetaldehyde, C7 to C16 alcohols, benzyl acetate, butyric acid, citric acid, isobutyl phenyl acetate, linalyl butyrate, malic acid, menthol, phenyl ethyl cinnamate, phenyl propyl formate, tannic acid, terpineol, vanillin, amyl salicylate, benzaldehyde, diphenyl ketone, indole, and the like.

### Single Chain Peptide-Based Reagents for Coupling a Benefit Agent to PMMA

The present peptide reagents comprising at least one of the present PMMA-binding peptides may be used in a composition to couple a benefit agent to surface, film, sheet, particle, bead, or microsphere comprising a surface having PMMA polymer. In a further embodiment, peptide reagent comprising a target binding domain (TBD) having affinity for a target surface, such as a body surface, may be used to couple a benefit agent comprising PMMA polymer to the target surface (i.e., the benefit agent comprises a surface of PMMA polymer capable of binding to the peptide reagent).

In one embodiment, the peptide reagents may contain one or more molecular linkers (L) separating the individual PMMA-binding peptides and/or separating the PMMA-binding peptide(s) or peptide-based reagent from the benefit agent or target binding domain (TBD).

As such, a peptide-based reagent is provided comprising the general structure:

([PBP]ₙ[L]ₓ-BA-[L]ₘ; or

([PBP]ₙ-[L]ₓ-TBD-[L]_{y})ₘ

wherein:
i) PBP is a polymethyl methacrylate-binding peptide;
ii) L is a linker molecule;
iii) BA is at least one benefit agent;
iv) TBD is a target binding domain;
v) x and y independently range from 0 to 10;
vi) n=1 to 10; and
vii) m=1 to 10;

wherein the polymethyl methacrylate-binding peptide comprises amino acid sequence SEQ ID NO: 14.

It may also be desirable to have multiple binding peptides coupled to the benefit agent to enhance the interaction between the peptide reagent and the surface comprising PMMA polymer. Either multiple copies of the same binding peptide or a combination of different binding peptides may be used. In the case of large particles, a large number of binding peptides, such as up to about 1,000 peptides, may be coupled to the particle. A smaller number of binding peptides can be coupled to smaller molecules, i.e., up to about 50.

### Linker Molecules

Linker molecules may optionally be used with one or more of the embodiments described herein. The linker may be any of a variety of molecules, such as alkyl chains, phenyl compounds, ethylene glycol, amides, esters and the like. Preferred linkers are hydrophilic and have a chain length from 1 to about 100 atoms, more preferably, from 2 to about 30 atoms. Examples of preferred linkers include, but are not limited to, ethanol amine, ethylene glycol, polyethylene with a chain length of 6 carbon atoms, polyethylene glycol with 3 to 6 repeating units, phenoxyethanol, propanolamide, butylene glycol, butyleneglycolamide, propyl phenyl, and ethyl, propyl, hexyl, steryl, cetyl, and palmitoyl alkyl chains. The linker may be covalently attached to the peptide and the benefit agent using any of the coupling chemistries described above. In order to facilitate incorporation of the linker, a bifunctional cross-linking agent that contains a linker and reactive groups at both ends for coupling to the peptide and the benefit agent may be used. Suitable bifunctional cross-linking agents are well known in the art and may include diamines, such as 1,6-diaminohexane; dialdehydes, such as glutaraldehyde; bis N-hydroxysuccinimide esters, such as ethylene glycol-bis(succinic acid N-hydroxysuccinimide ester), disuccinimidyl glutarate, disuccinimidyl suberate, and ethylene glycol-bis(succinimidylsuccinate); diisocyantes, such as hexamethylenediisocyanate; bis oxiranes, such as 1,4 butanediyl diglycidyl ether; dicarboxylic acids, such as succinyldisalicylate; and the like. Heterobifunctional cross-linking agents, which contain a different reactive group at each end, may also be used. Examples of peptide linkers are provided as SEQ ID NOs: 41, 253, and 254.

### Applications of PMMA-Binding Peptides

It will be appreciated by the skilled person that PMMA-binding peptides or peptide reagents comprising at least one of the present PMMA-binding peptides may be used in a multiplicity of formats including as delivery means for delivering benefits agents, in assays for diagnostic applications as well as in materials applications for coating PMMA polymer or copolymer surfaces. In one embodiment, a personal care composition comprising one or more of the present PMMA-binding peptides and/or peptide-based reagents is also provided to delivery (or enhance the durability of) a benefit agent to a body surface. Examples of personal care compositions may include coloring or conditioning compositions for the body surface described herein, such as hair, skin, nail, and/or tooth surfaces.

### EXAMPLES

It should be understood that these examples, while indicating various embodiments of the invention, are provided for illustration purposes. From the above discussion and the examples provided, one skilled in the art can ascertain the essential characteristics of this invention, and without departing from the spirit and scope thereof, can make various changes and modifications of the invention to adapt it to various uses and conditions.

The meaning of abbreviations used is as follows: “min” means minute(s), “sec” means second(s), “h” means hour(s), “μL” means microliter(s), “mL” means milliliter(s), “L” means liter(s), “nm” means nanometer(s), “mm” means millimeter(s), “cm” means centimeter(s), “μm” means micrometer(s), “mM” means millimolar, “M” means molar, “pmol” means picomole(s), “mmol” means millimole(s), “μmole” means micromole(s), “g” means gram(s), “μg” means microgram(s), “mg” means milligram(s), “g” means the gravitation constant, “rpm” means revolution(s) per minute, “pfu” means plaque forming unit, “BSA” means bovine serum albumin, “ELISA” means enzyme-linked immunosorbent assay, “A” means absorbance, “A₄₅₀” means the absorbance measured at a wavelength of 450 nm, “TBS” means Tris-buffered saline, “TBST-X” means Tris-buffered saline containing TWEEN® 20 (CAS# 9005-64-5) where “X” is the weight percent of TWEEN® 20, “vol % ” means volume percent, TRITON®-X100 is a detergent having CAS# 9002-93-1.

### General Methods:

Standard recombinant DNA and molecular cloning techniques used herein are well known in the art and are described by Sambrook, J. and Russell, D., Molecular Cloning: A Laboratory Manual, Third Edition, Cold Spring Harbor Laboratory Press, Cold Spring Harbor, N.Y. (2001); and by Silhavy, T. J., Bennan, M. L. and Enquist, L. W., Experiments with Gene Fusions, Cold Spring Harbor Laboratory Cold Press Spring Harbor, N.Y. (1984); and by Ausubel, F. M. et. al., Short Protocols in Molecular Biology, 5^{th} Ed. Current Protocols and John Wiley and Sons, Inc., N.Y., 2002.

Materials and methods suitable for the maintenance and growth of bacterial cultures are also well known in the art. Techniques suitable for use in the following Examples may be found in Manual of Methods for General Bacteriology, Phillipp Gerhardt, R. G. E. Murray, Ralph N. Costilow, Eugene W. Nester, Willis A. Wood, Noel R. Krieg and G. Briggs Phillips, eds., American Society for Microbiology, Washington, D.C., 1994, or by Thomas D. Brock in Biotechnology: A Textbook of Industrial Microbiology, Second Edition, Sinauer Associates, Inc., Sunderland, Mass., 1989.

All reagents and materials used for the growth and maintenance of bacterial cells were obtained from Aldrich Chemicals (Milwaukee, Wis.), BD Diagnostic Systems (Sparks, Md.), Life Technologies (Rockville, Md.), or Sigma Chemical Company (St. Louis, Mo.), unless otherwise specified.

### Example 1

### Selection of PMMA Polymer-Binding Peptides Using mRNA-Display Biopanning

The purpose of this Example is to demonstrate enrichment and isolation of PMMA-binding peptides using an mRNA display biopanning method.

mRNA-Display Peptide Libraries:

Methods to Make Libraries of DNA Molecules Suitable as Starting materials for mRNA-display are well-known in the art (see WO2005/051985). The following procedure was used to identify 27-mer peptides that have strong affinity for a PMMA polymer target material.

Briefly, a library of random nucleic acid molecules (dsDNA) each molecule encoding a peptide of desired length was generated. A linear peptide library containing 81 nucleotide positions or 27 randomized amino acid positions was used (“p27 library”). The p27 library was designed to include appropriate 5′ and 3′ regions for efficient in vitro transcription, translation, purification, and coupling to the MHA-oligonucleotide linker (MHA is 3′-[α-amino-p-methoxy-hydrocinnamido]-3′-deoxy-adenosine) in the individual molecules.

The DNA encoding the linear peptide library was designed to include a T7 promoter and a tobacco mosaic virus (TMV) translation initiation sequence operably linked to the coding sequence (CDS) (Liu et al., Methods in Enzymology, (2000) 318:268-293). The CDS was designed to encode: (1) a constant N-terminal flaking region comprising a hexa-histidine tag followed by a flexible linker (underlined) sequence (MHHHHHHSGSSSGSGSG; SEQ ID NO: 36), (2) the randomized 27-mer linear peptide, and (3) a constant C-terminal flanking region (TSGGSSGSSLGVASAI; SEQ ID NO: 37) comprising another flexible linker region (bold) and a C-terminal sequence optimized for efficient coupling to the MHA-oligonucleotide linker (double-underlined).

### In Vitro Transcription

Double stranded DNA (dsDNA) as result of the PCR reactions were transcribed into RNA using the RIBOMAX™ Express in vitro transcription kit (Promega Corp., Madison, Wis.). After incubation for at least 45 min at 37° C., DNase I was added and the incubation continued at 37° C. for additional 30 minutes to degrade all template DNA. The reaction mixture was purified by phenol/chloroform extraction. Then free nucleotides were removed by gel filtration using G25 microspin columns (Pharmacia Corp.; Milwaukee, Wis.). The concentration of purified RNA was determined by photometry at 260 nm.

### Library Preparation:

Approximately 10 pmol of highly purified RNA was produced by in vitro transcription from the p27 DNA library and purified after DNase I digestion (by phenol/chloroform extraction and gel filtration, methods described below). The 3′-end of the p27 library RNA was modified by attachment of a MHA-linker molecule (described above) and translated in vitro by means of a rabbit reticulocyte lysate. Covalent fusion products between peptide and coding RNA were purified on magnetic oligo(dT) beads, reverse transcribed, and again purified on a Ni-NTA purification matrix to remove uncoupled RNA and free peptides. About 8 pmol of peptide-RNA-cDNA-fusions were used as input for the first contact with target material during selection round 1.

### Chemical Coupling of RNA and MHA-Oligonucleotide Linker

Purified RNA was annealed (by heat denaturation for 1 minute at 85° C. and cooling down to 25° C. for 10 minutes) with a 1.5-fold excess of MHA-oligonucleotide linker-PEG₂A18 (5′-psoralen-UAG CGG AUG C A₁₈ (PEG-9)₂ CC-MHA [nucleotides shown in italics represent 2′-O-methyl-derivatives] (SEQ ID NO: 38). The covalent coupling was induced by radiation with UV-light (365 nm) for 15 min at room temperature. Aliquots of this reaction mixture before and after irradiation with UV were analyzed on a 6%-TBE-Urea-polyacrylamidgel to control the coupling efficiency (usually at least 60%).

### In Vitro Translation and ³⁵S-Labelling of Peptide-RNA Fusions

Ligated RNA was translated using a rabbit reticulocyte lysate from Promega in presence of 15 μCi ³⁵S-methionine (1000 Ci/mmole). After a 30 min incubation at 30° C., KCl and MgCl₂ were added to a final concentration of 530 mM and 150 mM respectively in order to promote formation of mRNA-peptide-fusions.

### Oligo(dT) Purification

For the purification of peptide-RNA-fusions from translation mixtures molecules were hybridized to magnetic oligo(dT) beads (Miltenyi Biotec; Bergisch Gladbach, Germany) in annealing buffer (100 mM Tris-HCl pH 8.0, 10 mM EDTA, 1 M NaCl and 0.25% TRITON® X-100) for 5 min at 4° C. Beads were separated from the mixture using magnetic-activated cell sorting (MiniMACS®-filtration columns; Miltenyi Biotec), repetitively washed with 100 mM Tris-HCl pH 8.0, 1 M NaCl, 0.25% TRITON® X-100 and finally eluted with water. A sample of this reaction was analyzed on 4-20% Tris/glycine-SDS-PAGE; radioactive bands were visualized using a Phosphorolmager.

### Reverse Transcription (RT)

The RNAs of Oligo(dT)-purified peptide-RNA-fusions were reverse transcribed using SUPERSCRIPT™ II Reverse Transcriptase (Invitrogen, Carlsbad, Calif.) according to the manufacturer's recommendations. RT reactions contained about 1.5-fold excess of 3′-reverse primer. A sample of this reaction was analyzed on 4-20% Tris/glycine-SDS-PAGE; radioactive bands were visualized using a PhosphorImager.

### His-Tag Purification

Reverse transcribed mRNA-peptide-fusion molecules were mixed with Ni-NTA-agarose (QIAGEN; Valencia, Calif.) in HBS buffer (20 mM HEPES (CAS # 7365-45-9) pH 7.0, 150 mM NaCl, 0.025% TRITON® X-100, 100 μg/mL sheared salmon sperm DNA, 1 mg/mL bovine serum albumin (BSA)) and incubated for 60 min at room temperature under gentle shaking. Ni-NTA was then filtrated and washed with HNT buffer (20 mM HEPES pH 7.0, 150 mM NaCl, 0.025% TRITON® X-100) containing 5 mM imidazole. Finally peptide-RNA-cDNA-fusions were eluted with 150 mM imidazole in HNT buffer (20 mM HEPES pH 7.0, 150 mM NaCl, 0.025% TRITON® X-100). A sample of this reaction was analyzed on 4-20% Tris/glycine-SDS-PAGE; radioactive bands were visualized using a PhosphorImager. BSA (final concentration 1 mg/mL) and sheared salmon sperm DNA (final concentration 100 μg/mL) were added to the eluates before contacting with target materials during selection step.

### Selection by Binding to Target Materials and Washing

A. Incubation of Peptide-RNA-cDNA-Fusion Library with Target Material:

Purified peptide-RNA-cDNA-fusions (PROFUSION™ molecules; Adnexus Therapeutics, Waltham, Mass.) after Ni-NTA purification were incubated for 60 minutes at room temperature in 1 mL (final volume) of 20 mM HEPES, pH 7.4, 150 mM NaCl, 1 mg/mL BSA, 100 μg/mL sheared salmon sperm DNA, 0.025% TRITON® X-100 in presence of DEPC-treated (diethylpyrocarbonate), blocked target material. Input activity of purified peptide-RNA-cDNA-fusions was determined by scintillation measurement.

### B. Washing:

Non-binding variants were washed away by one of the following washing procedures listed below:
Washing procedure A: used for washing the target material during selection round 1:
   5×5 sec. each with HNTriton buffer (20 mM HEPES, pH 7.4, 150 mM NaCl, 0.025% TRITON®-X100)
   1×5 sec 150 mM NaCl (for buffer removal before elution with KOH)
Washing procedure C: used for washing of target material during selection round 2-7:
   2×5 sec. each with HNTween buffer (20 mM HEPES, pH 7.4, 150 mM NaCl, 0.5% Tween-20)
   1×5 min. with 10% shampoo in HNTriton buffer
   1×5 sec with HNTween buffer including tube change
   1×5 min with 10% shampoo in HNTriton buffer
   3×5 sec with HNTween buffer; 1 tube change during the third wash
   1×5 sec 150 mM NaCl (for buffer removal before elution with KOH)
Washing procedure E: used for washing target material in round 8b:
   2×5 sec each with HNTween buffer (20 mM HEPES, pH 7.4, 150 mM NaCl, 0.5% TWEEN®-20)
   4×30 min. with 10% shampoo in HNTriton buffer
   1×5 sec with HNTween buffer including tube change
   3×5 sec with HNTween buffer; 1 tube change during the third wash
   1×5 sec 150 mM NaCl (for buffer removal before elution with KOH)
Washing procedure G: used for washing of target material in rounds 9b and 10b:
   2×5 sec each with HNTween buffer (20 mM HEPES, pH 7.4, 150 mM NaCl, 0.5% TWEEN®-20)
   1×5 min. with 10% shampoo in HNTriton buffer
   1×5 sec with HNTween buffer including tube change
   4×30 min with 10% shampoo in HNTriton buffer
   1×overnight with 10% shampoo in HNTriton buffer
   3×5 sec with HNTween buffer; 1 tube change during the third wash
   1×5 sec 150 mM NaCl (for buffer removal before elution with KOH)
      The shampoo used in the above washing procedures was a commercially available hair shampoo having the following composition:

| | |
|---|---|
| Water | 51% |
| Ammonium lauryl sulfate | 20% |
| Sodium lauryl ether sulfate | 15% |
| Cocamidopropyl betaine | 7% |
| Cocamide MEA | 2.5% |
| Miscellaneous minor components** | ~4.5% |

**(e.g. various pH adjusters, preservatives, vitamins, chelating agents, dispersants, lubricants, fragrances, and dyes)

### Comment on Incubation and Washing Conditions:

Normally during mRNA display selections a low detergent concentration is chosen to have low stringent conditions during up to 6 rounds of selection by keeping the detergent concentration at 0.025% TRITON®-X100. However, a higher stringency for the target material was applied from the beginning during incubation and washing (see washing procedures). The applied high concentrations of TWEEN®-20 and shampoo are close to the so called “critical micelle concentration” (CMC) allowing the formation of small micelles which might contain more than one peptide-RNA-cDNA-fusion. Since CMC driven aggregation of peptide-RNA-cDNA-fusions are critical for successful selections, higher concentrations of the detergents described above were not used.

cDNA Elution:

cdNAs of binding variants were eluted by incubation of target material in 50 μL of 100 mM KOH at 60° C. for 30 minutes. After centrifugation, supernatant was removed from target material and transferred into a fresh tube. KOH eluates were subsequently neutralized by addition of 1 μL of 1 M Tris/HCl, pH 7.0 and 3.8 μL of 1 M HCl (per 50 μL 100 mM KOH).

### Polymerase Chain Reaction (PCR):

After elution in KOH and neutralization, the recovered cDNAs were amplified by quantitative PCR with increasing numbers of amplification cycles (12, 15, 18, 21, 24 and 27 cycles). Products were subsequently analyzed by agarose gel electrophoresis over 2% agarose gels. Optimized conditions (minimal cycle number to get good enrichment of DNA of correct length) were then applied for a preparative PCR reaction and controlled again by agarose gel electrophoresis.

Analytical and preparative PCR reactions were performed in presence of 10 mM Tris-HCl (pH 8.8 at 25° C.), 50 mM KCl, 0.08% Nonidet P40, 2 mM MgCl₂, 2,5 mM dNTPs, 1 μM of each forward and reverse primer (5′-TAATACGACTCATAGGGACAATTACTATTTACAA TTACAATG-3′; SEQ ID NO: 39) and (5′-AATTAAATAGCGGATGCTACACCAAGACTAGAACCGCTG-3′; SEQ ID NO: 40), ⅕ volume of neutralized cDNA eluate and 0.05 U/μL Tag polymerase (Promega Corp.). Temperature program of PCR reaction is given below: Initial denaturation: 90 sec at 94° C.; cycling: 15 sec at 94° C. (denaturation), 20 sec at 60° C. (annealing), 30 sec at 72° C. (extension); post treatment: 3 min at 72° C. (post-treatment); hold at 4° C.

Enrichment of cDNA-RNA-Peptide Fusion Molecules Binding to PMMA

Ten rounds of selection were conducted and the relative binding of radioactively labeled cDNA-RNA-peptide fusion molecules to the PMMA polymer target material was measured. The amount of PMMA polymer (acrylic glass; PLEXIGLAS® VS100 [Altuglas International Arkema Inc., Philadelphia, Pa], used was once cylinder shaped PMMA pellet per selection divided into 4 quarters corresponding to approximately 27.4 mg.

Round 1 selection used washing procedure A as described above. Rounds 2-10 used various washing procedures with increased washing stringencies (see Table 1). The relative amount of enrichment (reported as percent enrichment of binding molecules relative to their respective input signals [activity of cDNA-RNA-peptide fusions before contacting with the target material]) is provided in Table 1.

TABLE 1
Relative binding of radioactive-labeled peptide-
RNA-cDNA-fusions on PMMA polymer during increasing
rounds of mRNA display selection:

| | | % Enrichment of cDNA- |
|---|---|---|
| | | RNA-peptide fusion |
| Selection | Washing | molecules having affinity |
| Round | Procedure | for PMMA polymer |
| R1 | A | 0.00 |
| R2 | C | 0.23 |
| R3 | C | 0.04 |
| R4 | C | 0.09 |
| R5 | C | 0.57 |
| R6 | C | 1.94 |
| R7 | C | 2.80^{a} |
| R8b | E | 2.80 |
| R9b | G | 1.47^{a} |
| R10b | G | 0.08^{a} |

^{a}= processed for sequencing

### Sequencing of 27-mer PMMA-Binding Peptides

The cDNA molecules from the enriched pool of PMMA-binding fusion molecules were isolated and PCR amplified as described above. The sequences of the DNA molecules encoding the PMMA-binding peptides isolated after rounds 7, 9b and 10b of selection were determined (˜30 samples each). The corresponding amino acid sequences of the PMMA-binding peptides are provided in Tables 2 (a-h). Several samples were identified encoding an identical or nearly identical amino acid sequence. Amino acid resides that vary between individual members with a specified group are in bold (Groups “A” through “G”).

TABLE 2a
Amino Acid Sequences of Selected PMMA-binding
Peptides Sharing Group “A” Structure

| Peptide | | | SEQ |
|---|---|---|---|
| Sample | Number | | ID |
| Numbers | Observed | Amino acid sequence | NO: |
| Round 7 | 17 | FLHGLIHGWYSLWMWMLSWPYM | 1 |
| samples: 15, | | VWWVF | |
| 19, 28, 21, | | | |
| 23, 27, 2, 6, | | | |
| 1, 9; | | | |
| Round 9b | | | |
| samples: 22, | | | |
| 10, 14, 21, | | | |
| 17, 9; | | | |
| Round 10b | | | |
| sample 6 | | | |
| Round 7 | 1 | FLHGLIHGWYSLWMWILSWPYM | 2 |
| sample 26 | | VWWVF | |
| Round 9b | 1 | FMHGLIHGWYSLWMWMLSWPYM | 3 |
| sample 4 | | VWWVF | |
| Round 7 | 1 | FLHGLIHGWYSLWMWMLSWPYM | 4 |
| sample 20 | | VWWVL | |
| Group “A” | — | FXHGLIHGWYSLWMWXLSWPYM | 5 |
| Sequence | | VWWVX | |

TABLE 2b
PMMA-binding Peptides Group “B” Structure

| Peptide | | | SEQ |
|---|---|---|---|
| Sample | Number | | ID |
| Numbers | Observed | Amino acid sequence | NO: |
| Round 7 | 8 | GWQRIWQSILCWMYFPLCLWMEWY | 6 |
| samples 16, | | RAI | |
| 17, 22, 8, | | | |
| 12, 7, and 3; | | | |
| Round 10b | | | |
| sample 5 | | | |
| Round 7 | 1 | GWQRIWQSILCWMYLPLCLWMEWY | 7 |
| sample 24 | | RAI | |
| Round 7 | 1 | GWQRIWQSIFCWMYFPLCLWMEWY | 8 |
| sample 25 | | RAI | |
| Group “B” | — | GWQRIWQSIXCWMYXPLCLWMEWY | 9 |
| Sequence | | RAI | |

TABLE 2c
PMMA-binding Peptides Group “C” Structure

| Peptide | | | SEQ |
|---|---|---|---|
| Sample | Number | | ID |
| Numbers | Observed | Amino acid sequence | NO: |
| Round 7 | 5 | GSETYLYWSWWWLYLWYWPFWY | 10 |
| samples 4, 5; | | MWAGM | |
| Round 9b | | | |
| samples 7, | | | |
| 15, 16 | | | |
| Round 10b | 1 | GSETYLYWSWWWLYLWYWPFWY | 11 |
| Sample 3 | | VWAGM | |
| Round 7 | 1 | GSETYLYWSWCWLYLWYWPFRY | 12 |
| Sample 13 | | MWAGM | |
| Round 7 | 1 | GSETYLYWSWWWLYSWYWPFWY | 13 |
| sample 10 | | MWAGM | |
| Group “C” | — | GSETYLYWSWXWLYXWYWPFXY | 14 |
| Sequence | | XWAGM | |

TABLE 2d
PMMA-binding Peptides Sharing Group “D”
Structure

| Peptide | | | SEQ |
|---|---|---|---|
| Sample | Number | | ID |
| Numbers | Observed | Amino acid sequence | NO: |
| Round 7 | 5 | MWHGLWLWMALYWWMTWSWFLW | 15 |
| Sample 30; | | PFRVI | |
| Round 9b | | | |
| Samples 11, | | | |
| 12, 8, 9 | | | |
| Round 9b | 2 | MWHGLWLWMALYWWMTWSWFLW | 16 |
| Sample 6; | | PFWVI | |
| Round 10b | | | |
| Sample 1 | | | |
| Round 10b | 1 | MWYGLWLWMALYWWMTWSWFLW | 17 |
| Sample 10 | | PFRVI | |
| Group “D” | — | MWXGLWLWMALYWWMTWSWFLW | 18 |
| Sequence | | PFXVI | |

TABLE 2e
PMMA-binding Peptides Sharing Group “E”
Structure

| Peptide | | | SEQ |
|---|---|---|---|
| Sample | Number | | ID |
| Numbers | Observed | Amino acid sequence | NO: |
| Round 9b | 1 | IGWWLLRYWLYLQWKLYVWWFS | 19 |
| Sample 19 | | VLWTF | |
| Round 9b | 1 | IGWWLLRYWLYLQWKLYAWWFS | 20 |
| Sample 8 | | VLWTF | |
| Group “E” | — | IGWWLLRYWLYLQWKLYXWWFS | 21 |
| Sequence | | VLWTF | |

TABLE 2f
PMMA-binding Peptides Sharing Group “F”
Structure

| Peptide | | | SEQ |
|---|---|---|---|
| Sample | Number | | ID |
| Numbers | Observed | Amino acid sequence | NO: |
| Round 9 | 2 | RLDSWIFQTWLMWIWMVWPWLWPF | 22 |
| Samples 3, 20 | | WWL | |
| Round 10b | 1 | LDSWIFQTWLMWIWMVWPWLWPF | 23 |
| Sample 4 | | WWL | |
| Group “F” | — | XLDSWIFQTWLMWIWMVWPWLWPF | 24 |
| Sequence | | WWL | |

TABLE 2g
PMMA-binding Peptides Sharing Group “G”
Structure

| Peptide | | | SEQ |
|---|---|---|---|
| Sample | Number | | ID |
| Numbers | Observed | Amino acid sequence | NO: |
| S15R9b_2 | 1 | VWWTFFGWFYWLWAMWWNVSLA | 25 |
| | | LWEWV | |
| S15R10b_7 | 1 | VWWAFFGWFYWLWAMWWNVSLA | 26 |
| | | LWEWV | |
| Group “G” | — | VWWXFFGWFYWLWAMWWNVSLA | 27 |
| Sequence | | LWEWV | |

TABLE 2h
Additional PMMA-binding Peptides

| Peptide | | | SEQ |
|---|---|---|---|
| Sample | Number | | ID |
| Numbers | Observed | Amino acid sequence | NO: |
| Round 7 | 1 | VVPLMWVYWWVFMWGWPMVFWYTWWAA | 28 |
| Sample 11 | | | |
| Round 7 | 1 | FSSRDFLGYWISWLMWPYFVLWRWLMH | 29 |
| Sample 14 | | | |
| Round 7 | 1 | RSGLKRKVLRHVWTVMWTMGSWLHGSL | 30 |
| Sample 29 | | | |
| Round 9b | 1 | FWYGLWLFWWWHTWFVWRTLWYWMVWL | 31 |
| Sample 18 | | | |
| Round 9b | 1 | TLWTFYWLSWAWYMPLWPWWLYWLMWG | 32 |
| Sample 13 | | | |
| Round 9b | 1 | LLWLFWWPWLWWLCVTQWSYEMGMGWW | 33 |
| Sample 15 | | | |
| Round 9b | 1 | HFWAWYIEWLRWYLYVPLWFRWFVGF | 34 |
| Sample 1 | | | |
| Round 10b | 1 | YVLFFMAVWWPWWLLMWIWQNLMTMTT | 35 |
| Sample 2 | | | |

### Example 2

### Confirmation of PMMA-Binding Affinity

The purpose of this Example was to confirm the affinity of at least one PMMA-binding peptide identified by mRNA-display for a PMMA resin surface, measured as MB₅₀ values, using an ELISA assay to confirm that selection process produced PMMA-binding peptides with strong affinity for PMMA polymer resin.

The peptide was synthesized using a standard solid phage synthesis method and was biotinylated by adding a biotinylated lysine residue at the C-terminus of the amino acid binding sequence for detection purposes. The peptide tested was SEQ ID NO: 6 (referred to herein as “CPXB”). A C-terminal biotinylated lysine was added to the SEQ ID NO: 6; provided separately herein as SEQ ID NO: 257.

The MB₅₀ measurement of the biotinylated peptide binding to PMMA was done using PMMA polymer resin from Bangs Laboratories, Inc. The polymethyl methacrylate (dry) particles were 140 μm in diameter. Each teat-tube contains 1 mg of the particles and three tubes for peptide concentration (from 1 to 1,000 μM). The PMMA resin sample was incubated in SUPERBLOCK® blocking buffer (Pierce Chemical) for 1 hour at room temperature (˜22° C.), followed by 3 washes with TBST (TBS in 0.05% TWEEN® 20). Peptide binding buffer consisting of various concentrations of biotinylated peptide in TBST and 1 mg/mL BSA was added to the PMMA polymer samples and incubated for 1 hour at room temperature, followed by 6 TBST washes. Then, the streptavidin-horseradish peroxidase (HRP) conjugate (Pierce Chemical Co., Rockford, Ill.) was added to each well (1.0 μg per well), and incubated for 1 h at room temperature, followed by 6 times of washes with TBST. All samples were transferred to new tubes and the chromogenic agent ABTS (2,2′-azino-bis(3-ethylbenzthiazoline-6-sulphonic acid)) was added. The color development and the absorbance measurements were performed following the manufacturer's protocol. The plates were read at A₄₀₅ nm. The results were plotted as A₄₀₅ versus the concentration of peptide using GraphPad Prism 4.0 (GraphPad Software, Inc., San Diego, Calif.). The MB₅₀ value was calculated from Scatchard plots and is shown Table 3.

The very low MB₅₀ value measured for CPXB verifies that PMMA-binding peptide sequences identified by mRNA-display should have strong affinity for PMMA resin.

TABLE 3
MB₅₀ Value for PMMA-binding peptide CPXB

| Peptide | | |
|---|---|---|
| ID | | |
| NO. | Peptide Sequence | MB₅₀ (M) |
| CXPB | PLWRRITKRKLVRPVATLMWYWFTS | 2.5 × 10⁻⁸ |
| | KRK-(biotin)-NH₂ | |
| | (SEQ ID NO: 257) | |

What is claimed is:

1. An isolated peptide having affinity for polymethyl methacrylate polymer (PMMA-binding peptide), said peptide having the general structure:

| | |
|---|---|
| GSETYLYWSWX₁WLYX₂WYWPFX₃YX₄WAGM | (SEQ ID NO: 14) |

wherein
i) X₁ is W or C;
ii) X₂ is L or S;
iii) X₃ is W or R; and
iv) X₄ is V or M.

2. A peptide-based reagent having a general structure selected from the group consisting of:
([PBP]ₙ-[L]ₓ-BA-[L]_{y})ₘ; and
([PBP]ₙ-Mₓ-TBD-[L]_{y})ₘ
wherein:
i) PBP is the peptide of claim 1 having affinity for polymethyl methacrylate polymer;
ii) L is a linker molecule;
iii) BA is at least one benefit agent;
iv) AD is at least one active domain;
v) TBD is a target binding domain;
vi) x and y are independently 0 or 1;
vii) n=1 to 10; and
viii) m=1 to 10.

3. The peptide-based reagent according to claim 2 wherein the linker molecule is a peptide linker ranging form 1 to 50 amino acids in length.

4. The peptide-based reagent according to claim 2 wherein the benefit agent is selected from the group consisting of pharmaceuticals, markers, colorants, conditioners, fragrances, and antimicrobial agents.

5. The peptide-based reagent according to claim 2 wherein the target binding domain is a body surface binding domain comprising at least one peptide having affinity for a body surface selected from the group consisting of hair, skin, nails and teeth.

6. A method for binding a peptide-based reagent to polymethyl methacrylate (PMMA) comprising:
a) providing the peptide-based reagent according to claim 2: and
b) contacting the peptide-based reagent of (a) with a surface comprising PMMA whereby the peptide reagent binds to the PMMA.

7. A personal care composition comprising the peptide of claim 1 or the peptide-based reagent of claim 2.


SEQUENCE LISTING (474 sequences)

SEQ ID NO: 1 - protein, 27 aa, artificial sequence
  synthetic construct
FLHGLIHGWYSLWMWMLSWPYMVWWVF

SEQ ID NO: 2 - protein, 27 aa, artificial sequence
  synthetic construct
FLHGLIHGWYSLWMWILSWPYMVWWVF

SEQ ID NO: 3 - protein, 27 aa, artificial sequence
  synthetic construct
FMHGLIHGWYSLWMWMLSWPYMVWWVF

SEQ ID NO: 4 - protein, 27 aa, artificial sequence
  synthetic construct
FLHGLIHGWYSLWMWMLSWPYMVWWVL

SEQ ID NO: 5 - protein, 27 aa, artificial sequence
  synthetic construct
FXHGLIHGWYSLWMWXLSWPYMVWWVX

SEQ ID NO: 6 - protein, 27 aa, artificial sequence
  synthetic construct
GWQRIWQSILCWMYFPLCLWMEWYRAI

SEQ ID NO: 7 - protein, 27 aa, artificial sequence
  synthetic construct
GWQRIWQSILCWMYLPLCLWMEWYRAI

SEQ ID NO: 8 - protein, 27 aa, artificial sequence
  synthetic construct
GWQRIWQSIFCWMYFPLCLWMEWYRAI

SEQ ID NO: 9 - protein, 27 aa, artificial sequence
  synthetic construct
GWQRIWQSIXCWMYXPLCLWMEWYRAI

SEQ ID NO: 10 - protein, 27 aa, artificial sequence
  synthetic construct
GSETYLYWSWWWLYLWYWPFWYMWAGM

SEQ ID NO: 11 - protein, 27 aa, artificial sequence
  synthetic construct
GSETYLYWSWWWLYLWYWPFWYVWAGM

SEQ ID NO: 12 - protein, 27 aa, artificial sequence
  synthetic construct
GSETYLYWSWCWLYLWYWPFRYMWAGM

SEQ ID NO: 13 - protein, 27 aa, artificial sequence
  synthetic construct
GSETYLYWSWWWLYSWYWPFWYMWAGM

SEQ ID NO: 14 - protein, 27 aa, artificial sequence
  synthetic construct
GSETYLYWSWXWLYXWYWPFXYXWAGM

SEQ ID NO: 15 - protein, 27 aa, artificial sequence
  synthetic construct
MWHGLWLWMALYWWMTWSWFLWPFRVI

SEQ ID NO: 16 - protein, 27 aa, artificial sequence
  synthetic construct
MWHGLWLWMALYWWMTWSWFLWPFWVI

SEQ ID NO: 17 - protein, 27 aa, artificial sequence
  synthetic construct
MWYGLWLWMALYWWMTWSWFLWPFRVI

SEQ ID NO: 18 - protein, 27 aa, artificial sequence
  synthetic construct
MWXGLWLWMALYWWMTWSWFLWPFXVI

SEQ ID NO: 19 - protein, 27 aa, artificial sequence
  synthetic construct
IGWWLLRYWLYLQWKLYVWWFSVLWTF

SEQ ID NO: 20 - protein, 27 aa, artificial sequence
  synthetic construct
IGWWLLRYWLYLQWKLYAWWFSVLWTF

SEQ ID NO: 21 - protein, 27 aa, artificial sequence
  synthetic construct
IGWWLLRYWLYLQWKLYXWWFSVLWTF

SEQ ID NO: 22 - protein, 27 aa, artificial sequence
  synthetic construct
RLDSWIFQTWLMWIWMVWPWLWPFWWL

SEQ ID NO: 23 - protein, 26 aa, artificial sequence
  synthetic construct
LDSWIFQTWLMWIWMVWPWLWPFWWL

SEQ ID NO: 24 - protein, 27 aa, artificial sequence
  synthetic construct
XLDSWIFQTWLMWIWMVWPWLWPFWWL

SEQ ID NO: 25 - protein, 27 aa, artificial sequence
  synthetic construct
VWWTFFGWFYWLWAMWWNVSLALWEWV

SEQ ID NO: 26 - protein, 27 aa, artificial sequence
  synthetic construct
VWWAFFGWFYWLWAMWWNVSLALWEWV

SEQ ID NO: 27 - protein, 27 aa, artificial sequence
  synthetic construct
VWWXFFGWFYWLWAMWWNVSLALWEWV

SEQ ID NO: 28 - protein, 27 aa, artificial sequence
  synthetic construct
VVPLMWVYWWVFMWGWPMVFWYTWWAA

SEQ ID NO: 29 - protein, 27 aa, artificial sequence
  synthetic construct
FSSRDFLGYWISWLMWPYFVLWRWLMH

SEQ ID NO: 30 - protein, 27 aa, artificial sequence
  synthetic construct
RSGLKRKVLRHVWTVMWTMGSWLHGSL

SEQ ID NO: 31 - protein, 27 aa, artificial sequence
  synthetic construct
FWYGLWLFWWWHTWFVWRTLWYWMVWL

SEQ ID NO: 32 - protein, 27 aa, artificial sequence
  synthetic construct
TLWTFYWLSWAWYMPLWPWWLYWLMWG

SEQ ID NO: 33 - protein, 27 aa, artificial sequence
  synthetic construct
LLWLFWWPWLWWLCVTQWSYEMGMGWW

SEQ ID NO: 34 - protein, 27 aa, artificial sequence
  synthetic construct
HFWAWYIEWLRWYLYVPLVVFRWFVGF

SEQ ID NO: 35 - protein, 27 aa, artificial sequence
  synthetic construct
YVLFFMAVWWPWWLLMWIWQNLMTMTT

SEQ ID NO: 36 - protein, 17 aa, artificial sequence
  N terminal constant region - synthetic
MHHHHHHSGSSSGSGSG

SEQ ID NO: 37 - protein, 16 aa, artificial sequence
  C terminal constant region - synthetic
TSGGSSGSSLGVASAI

SEQ ID NO: 38 - RNA, 28 nt, artificial sequence
  Synthetic oligonucleotide linker
uagcggaugcaaaaaaaaaaaaaaaaaa

SEQ ID NO: 39 - DNA, 42 bp, artificial sequence
  primer
taatacgactcatagggacaattactatttacaattacaatg

SEQ ID NO: 40 - DNA, 39 bp, artificial sequence
  primer
aattaaatagcggatgctacaccaagactagaaccgctg

SEQ ID NO: 41 - protein, 8 aa, Artificial Sequence
  Caspase 3 cleavage site
LESGDEVD

SEQ ID NO: 42 - protein, 12 aa, Artificial sequence
  Polymethylmethacrylate binding peptides
IPWWNIRAPLNA

SEQ ID NO: 43 - protein, 12 aa, Artificial Sequence
  Polymethylmethacrylate-binding peptide
TAVMNVVNNQLS

SEQ ID NO: 44 - protein, 12 aa, Artificial Sequence
  Polymethylmethacrylate-binding peptide
VPWWAPSKLSMQ

SEQ ID NO: 45 - protein, 12 aa, Artificial Sequence
  Polymethylmethacrylate-binding peptide
MVMAPHTPRARS

SEQ ID NO: 46 - protein, 12 aa, Artificial Sequence
  Polymethylmethacrylate-binding peptide
TYPNWAHLLSHY

SEQ ID NO: 47 - protein, 7 aa, Artificial Sequence
  Polymethylmethacrylate-binding peptide
TPWWRIT

SEQ ID NO: 48 - protein, 7 aa, Artificial Sequence
  Polymethylmethacrylate-binding peptide
DLTLPFH

SEQ ID NO: 49 - protein, 7 aa, Artificial Sequence
  Polymethylmethacrylate-binding peptide
GTSIPAM

SEQ ID NO: 50 - protein, 7 aa, Artificial Sequence
  Polymethylmethacrylate-binding peptide
HHKHVVA

SEQ ID NO: 51 - protein, 7 aa, Artificial Sequence
  Polymethylmethacrylate-binding peptide
HHHKHFM

SEQ ID NO: 52 - protein, 7 aa, Artificial Sequence
  Polymethylmethacrylate-binding peptide
HHHRHQG

SEQ ID NO: 53 - protein, 7 aa, Artificial Sequence
  Polymethylmethacrylate-binding peptide
HHWHAPR

SEQ ID NO: 54 - protein, 12 aa, Artificial Sequence
  PMMA-binding peptide
APWHLSSQYSGT

SEQ ID NO: 55 - protein, 14 aa, Artificial Sequence
  PMMA-binding peptide
GYCLRVDEPTVCSG

SEQ ID NO: 56 - protein, 15 aa, Artificial Sequence
  PMMA-binding peptide
HIHPSDNFPHKNRTH

SEQ ID NO: 57 - protein, 20 aa, Artificial Sequence
  PMMA-binding peptide
HTHHDTHKPWPTDDHRNSSV

SEQ ID NO: 58 - protein, 20 aa, Artificial Sequence
  PMMA-binding peptide
PEDRPSRTNALHHNAHHHNA

SEQ ID NO: 59 - protein, 15 aa, Artificial Sequence
  PMMA-binding peptide
TPHNHATTNHHAGKK

SEQ ID NO: 60 - protein, 15 aa, Artificial Sequence
  PMMA-binding peptide
EMVKDSNQRNTRISS

SEQ ID NO: 61 - protein, 12 aa, Artificial Sequence
  PMMA-binding peptide
HYSRYNPGPHPL

SEQ ID NO: 62 - protein, 12 aa, Artificial Sequence
  PMMA-binding peptide
IDTFYMSTMSHS

SEQ ID NO: 63 - protein, 20 aa, Artificial Sequence
  PMMA-binding peptide
PMKEATHPVPPHKHSETPTA

SEQ ID NO: 64 - protein, 12 aa, Artificial Sequence
  PMMA-binding peptide
YQTSSPAKQSVG

SEQ ID NO: 65 - protein, 15 aa, Artificial Sequence
  PMMA-binding peptide
HLPSYQITQTHAQYR

SEQ ID NO: 66 - protein, 20 aa, Artificial Sequence
  PMMA-binding peptide
TTPKTTYHQSRAPVTAMSEV

SEQ ID NO: 67 - protein, 15 aa, Artificial Sequence
  PMMA-binding peptide
DRIHHKSHHVTTNHF

SEQ ID NO: 68 - protein, 12 aa, Artificial Sequence
  PMMA-binding peptide
WAPEKDYMQLMK

SEQ ID NO: 69 - protein, 12 aa, Artificial sequence
  Polypropylene-binding peptides
TSDIKSRSPHHR

SEQ ID NO: 70 - protein, 12 aa, Artificial Sequence
  Polypropylenebinding peptide
HTQNMRMYEPWF

SEQ ID NO: 71 - protein, 7 aa, Artificial Sequence
  Polypropylene-binding peptide
LPPGSLA

SEQ ID NO: 72 - protein, 12 aa, Artificial Sequence
  Polypropylene-binding peptide
MPAVMSSAQVPR

SEQ ID NO: 73 - protein, 12 aa, Artificial Sequence
  Polypropylene-binding peptide
NQSFLPLDFPFR

SEQ ID NO: 74 - protein, 12 aa, Artificial Sequence
  Polypropylene-binding peptide
SILSTMSPHGAT

SEQ ID NO: 75 - protein, 12 aa, Artificial Sequence
  Polypropylene-binding peptide
SMKYSHSTAPAL

SEQ ID NO: 76 - protein, 12 aa, Artificial sequence
  Polytetrafluoroethylene-binding peptides
ESSYSWSPARLS

SEQ ID NO: 77 - protein, 12 aa, Artificial Sequence
  Polytetrafluoroethylene-binding peptide
GPLKLLHAWWQP

SEQ ID NO: 78 - protein, 7 aa, Artificial Sequence
  Polytetrafluoroethylene-binding peptide
NALTRPV

SEQ ID NO: 79 - protein, 7 aa, Artificial Sequence
  Polytetrafluoroethylene-binding peptide
SAPSSKN

SEQ ID NO: 80 - protein, 12 aa, Artificial Sequence
  Polytetrafluoroethylene-binding peptide
SVSVGMKPSPRP

SEQ ID NO: 81 - protein, 12 aa, Artificial Sequence
  Polytetrafluoroethylene-binding peptide
SYYSLPPIFHIP

SEQ ID NO: 82 - protein, 12 aa, Artificial Sequence
  Polytetrafluoroethylene-binding peptide
TFTPYSITHALL

SEQ ID NO: 83 - protein, 12 aa, Artificial Sequence
  Polytetrafluoroethylene-binding peptide
TMGFTAPRFPHY

SEQ ID NO: 84 - protein, 12 aa, Artificial Sequence
  Polytetrafluoroethylene-binding peptide
TNPFPPPPSSPA

SEQ ID NO: 85 - protein, 12 aa, Artificial sequence
  Polyethylene-binding peptides
HNKSSPLTAALP

SEQ ID NO: 86 - protein, 12 aa, Artificial Sequence
  Polyethylene-binding peptide
LPPWKHKTSGVA

SEQ ID NO: 87 - protein, 12 aa, Artificial Sequence
  Polyethylene-binding peptide
LPWWLRDSYLLP

SEQ ID NO: 88 - protein, 12 aa, Artificial Sequence
  Polyethylene-binding peptide
VPWWKHPPLPVP

SEQ ID NO: 89 - protein, 12 aa, Artificial Sequence
  Polyethylene-binding peptide
HHKQWHNHPHHA

SEQ ID NO: 90 - protein, 12 aa, Artificial Sequence
  Polyethylene-binding peptide
HIFSSWHQMWHR

SEQ ID NO: 91 - protein, 12 aa, Artificial Sequence
  Polyethylene-binding peptide
WPAWKTHPILRM

SEQ ID NO: 92 - protein, 7 aa, Artificial sequence
  Nylon-binding peptides
KTPPTRP

SEQ ID NO: 93 - protein, 7 aa, Artificial Sequence
  Nylon-binding peptide
VINPNLD

SEQ ID NO: 94 - protein, 7 aa, Artificial Sequence
  Nylon-binding peptide
KVWIVST

SEQ ID NO: 95 - protein, 7 aa, Artificial Sequence
  Nylon-binding peptide
AEPVAML

SEQ ID NO: 96 - protein, 7 aa, Artificial Sequence
  Nylon-binding peptide
AELVAML

SEQ ID NO: 97 - protein, 7 aa, Artificial Sequence
  Nylon-binding peptide
HSLRLDW

SEQ ID NO: 98 - protein, 13 aa, Artificial sequence
  Polystyrene-binding peptide
TSTASPTMQSKIR

SEQ ID NO: 99 - protein, 12 aa, Artificial Sequence
  Polystyrene-binding peptide
KRNHWQRMHLSA

SEQ ID NO: 100 - protein, 12 aa, Artificial Sequence
  Polystyrene-binding peptide
SHATPPQGLGPQ

SEQ ID NO: 101 - protein, 20 aa, artificial sequence
  cellulose acetate-binding peptide
ATTPPSGKAAAHSAARQKGN

SEQ ID NO: 102 - protein, 15 aa, artificial sequence
  cellulose acetate-binding peptide
DTIHPNKMKSPSSPL

SEQ ID NO: 103 - protein, 20 aa, artificial sequence
  cellulose aceteate-binding peptide
NGNNHTDIPNRSSYTGGSFA

SEQ ID NO: 104 - protein, 15 aa, ARTIFICIAL SEQUENCE
  cellulose acetate-binding peptide
SDETGPQIPHRRPTW

SEQ ID NO: 105 - protein, 7 aa, Artificial Sequence
  Pigment-binding peptide
MPPPLMQ

SEQ ID NO: 106 - protein, 7 aa, Artificial Sequence
  Pigment-binding peptide
FHENWPS

SEQ ID NO: 107 - protein, 12 aa, Artificial Sequence
  Pigment-binding peptide
RTAPTTPLLLSL

SEQ ID NO: 108 - protein, 12 aa, Artificial Sequence
  Pigment-binding peptide
WHLSWSPVPLPT

SEQ ID NO: 109 - protein, 7 aa, Artificial Sequence
  Pigment-binding peptide
PHARLVG

SEQ ID NO: 110 - protein, 7 aa, Artificial Sequence
  Pigment-binding peptide
NIPYHHP

SEQ ID NO: 111 - protein, 7 aa, Artificial Sequence
  Pigment-binding peptide
TTMPAIP

SEQ ID NO: 112 - protein, 7 aa, Artificial Sequence
  Pigment-binding peptide
HNLPPRS

SEQ ID NO: 113 - protein, 12 aa, Artificial Sequence
  Pigment-binding peptide
AHKTQMGVRQPA

SEQ ID NO: 114 - protein, 12 aa, Artificial Sequence
  Pigment-binding peptide
ADNVQMGVSHTP

SEQ ID NO: 115 - protein, 12 aa, Artificial Sequence
  Pigment-binding peptide
AHNAQMGVSHPP

SEQ ID NO: 116 - protein, 12 aa, Artificial Sequence
  Pigment-binding peptide
ADYVGMGVSHRP

SEQ ID NO: 117 - protein, 12 aa, Artificial Sequence
  Pigment-binding peptide
SVSVGMKPSPRP

SEQ ID NO: 118 - protein, 7 aa, Artificial Sequence
  Pigment-binding peptide
YPNTALV

SEQ ID NO: 119 - protein, 7 aa, Artificial Sequence
  Pigment-binding peptide
VATRIVS

SEQ ID NO: 120 - protein, 12 aa, Artificial Sequence
  Pigment-binding peptide
HSLKNSMLTVMA

SEQ ID NO: 121 - protein, 7 aa, Artificial Sequence
  Pigment-binding peptide
NYPTQAP

SEQ ID NO: 122 - protein, 7 aa, Artificial Sequence
  Pigment-binding peptide
KCCYSVG

SEQ ID NO: 123 - protein, 12 aa, Artificial Sequence
  Pigment-binding peptide
RHDLNTWLPPVK

SEQ ID NO: 124 - protein, 12 aa, artificial sequence
  synthetic construct
EISLPAKLPSAS

SEQ ID NO: 125 - protein, 12 aa, Artificial Sequence
  Pigment-binding peptide
SDYVGMRPSPRH

SEQ ID NO: 126 - protein, 12 aa, Artificial Sequence
  Pigment-binding peptide
SDYVGMRLSPSQ

SEQ ID NO: 127 - protein, 12 aa, Artificial Sequence
  Pigment-binding peptide
SVSVGIQPSPRP

SEQ ID NO: 128 - protein, 12 aa, Artificial Sequence
  Pigment-binding peptide
YVSVGIKPSPRP

SEQ ID NO: 129 - protein, 12 aa, Artificial Sequence
  Pigment-binding peptide
YVCEGIHPCPRP

SEQ ID NO: 130 - protein, 12 aa, artificial sequence
  synthetic pigment binding peptide
WAPEKDHMQLMK

SEQ ID NO: 131 - protein, 12 aa, artificial sequence
  Iron oxide-binding peptide
WAPEKDYMQLMK

SEQ ID NO: 132 - protein, 20 aa, artificial sequence
  Iron oxide-binding peptide
CPLDTPTHKTKHEYKTRCRH

SEQ ID NO: 133 - protein, 18 aa, artificial sequence
  Iron oxide-binding peptide
DHDHPRLHKRQEKSEHLH

SEQ ID NO: 134 - protein, 20 aa, artificial sequence
  Iron oxide-binding peptide
DSHHNHHKQDSRPQHRKTPN

SEQ ID NO: 135 - protein, 15 aa, artificial sequence
  Iron oxide-binding peptide
EGGNAPHHKPHHRKH

SEQ ID NO: 136 - protein, 18 aa, artificial sequence
  Iron oxide-binding peptide
HDSHRPLTQHGHRHSHVP

SEQ ID NO: 137 - protein, 18 aa, artificial sequence
  Iron oxide-binding peptide
HDSNHCSHSTRRPNCART

SEQ ID NO: 138 - protein, 15 aa, artificial sequence
  Iron oxide-binding peptide
ATRVDNTPASNPPSL

SEQ ID NO: 139 - protein, 15 aa, artificial sequence
  Iron oxide-binding peptide
DGIKPFHLMTPTLAN

SEQ ID NO: 140 - protein, 18 aa, artificial sequence
  Iron oxide-binding peptide
DITPPGSTHHRKPHRHQH

SEQ ID NO: 141 - protein, 15 aa, artificial sequence
  Iron oxide-binding peptide
DNLWPQPLNVEDDRY

SEQ ID NO: 142 - protein, 18 aa, artificial sequence
  Iron oxide-binding peptide
ENEKHRHNTHEALHSHFK

SEQ ID NO: 143 - protein, 20 aa, artificial sequence
  Iron oxide-binding peptide
GAIWPASSALMTEHNPTDNH

SEQ ID NO: 144 - protein, 15 aa, artificial sequence
  Iron oxide-binding peptide
GDTNQDTVMWYYTVN

SEQ ID NO: 145 - protein, 15 aa, artificial sequence
  Iron oxide-binding peptide
HNGPYGMLSTGKIHF

SEQ ID NO: 146 - protein, 15 aa, artificial sequence
  Iron oxide-binding peptide
LDGGYRDTPDNYLKG

SEQ ID NO: 147 - protein, 15 aa, artificial sequence
  Iron oxide-binding peptide
LHTKTENSHTNMKTT

SEQ ID NO: 148 - protein, 20 aa, artificial sequence
  Iron oxide-binding peptide
NAQYDPPTLNKGAVRKAAST

SEQ ID NO: 149 - protein, 15 aa, artificial sequence
  Iron oxide-binding peptide
NGNNHTDIPNRSSYT

SEQ ID NO: 150 - protein, 18 aa, artificial sequence
  Iron oxide-binding peptide
QSTNHHHPHAKHPRVNTH

SEQ ID NO: 151 - protein, 15 aa, artificial sequence
  Iron oxide-binding peptide
SNNDYVGTYPATAIQ

SEQ ID NO: 152 - protein, 15 aa, artificial sequence
  Iron oxide-binding peptide
STQHNLHDRNIYFVS

SEQ ID NO: 153 - protein, 20 aa, artificial sequence
  Iron oxide-binding peptide
TANNKTPAGAPNAAVGLAQR

SEQ ID NO: 154 - protein, 15 aa, artificial sequence
  Iron oxide-binding peptide
TEPTRISNYRSIPND

SEQ ID NO: 155 - protein, 18 aa, artificial sequence
  Iron oxide-binding peptide
THNPREHARHHHHNEYKH

SEQ ID NO: 156 - protein, 15 aa, artificial sequence
  Iron oxide-binding peptide
THPPCWYETNCIVQE

SEQ ID NO: 157 - protein, 20 aa, artificial sequence
  Iron oxide-binding peptide
TTNPHKPASHHHDHRPALRH

SEQ ID NO: 158 - protein, 15 aa, artificial sequence
  Iron oxide-binding peptide
WLVADNATDGHSHQK

SEQ ID NO: 159 - protein, 15 aa, artificial sequence
  Iron oxide-binding peptide
YTDSMSDQTPEFAKY

SEQ ID NO: 160 - protein, 27 aa, artificial sequence
  Clay-binding peptide
GHGSPSNSHHGSKKCDMGNSRAKCKRL

SEQ ID NO: 161 - protein, 27 aa, artificial sequence
  Clay-binding peptide
SDRHNLRNSWSISRHCRRKQGRCLPAH

SEQ ID NO: 162 - protein, 27 aa, artificial sequence
  Clay-binding peptide
KKSNKGHHPSSKGKGPPWSEWDKKNGP

SEQ ID NO: 163 - protein, 27 aa, artificial sequence
  Clay-binding peptide
KKSNKGPHPSSKGKGPPWSEWDKKNGP

SEQ ID NO: 164 - protein, 22 aa, artificial sequence
  Clay-binding peptide
VGRHHSKAKQKRPHGGKGQNKN

SEQ ID NO: 165 - protein, 22 aa, artificial sequence
  Clay-binding peptide
VGRHHPKAKQKRPHGGKGQNKN

SEQ ID NO: 166 - protein, 17 aa, artificial sequence
  Clay-binding peptide
GRRPRARGRSRRGSTKT

SEQ ID NO: 167 - protein, 19 aa, artificial sequence
  Clay-binding peptide
LGVIRNHVVRGRRHHQHVR

SEQ ID NO: 168 - protein, 27 aa, artificial sequence
  Clay-binding peptide
QPGRPTEVHPELVRKSAYLVNPSEDIR

SEQ ID NO: 169 - protein, 27 aa, artificial sequence
  Clay-binding peptide
HRSEKPKNVKYKRGYWERGNQKKHGPG

SEQ ID NO: 170 - protein, 27 aa, artificial sequence
  Clay-binding peptide
GSHKRRGSYALLRTRGVGRQAELEHLL

SEQ ID NO: 171 - protein, 27 aa, artificial sequence
  Clay-binding peptide
VGEKPRRKSKGAKAKKARTKEEKLPKN

SEQ ID NO: 172 - protein, 27 aa, artificial sequence
  Clay-binding peptide
NKGHKQSGSPRHSNKKEKKTQQKRGQP

SEQ ID NO: 173 - protein, 27 aa, artificial sequence
  Clay-binding peptide
HWGSQHKTGLRNHKRSRRDSLGKRGTD

SEQ ID NO: 174 - protein, 27 aa, artificial sequence
  Clay-binding peptide
KGWGSSSGPPGLTGKALGKGRLKPKKK

SEQ ID NO: 175 - protein, 27 aa, artificial sequence
  Calcium carbonate binding peptide
RNNKGSKKVDDKRRKTVHNTKSRAKYS

SEQ ID NO: 176 - protein, 27 aa, artificial sequence
  Calcium carbonate binding peptide
RNNKGSKKVDDKRRKTVHNTKSRAKHS

SEQ ID NO: 177 - protein, 27 aa, artificial sequence
  Calcium carbonate binding peptide
RDNKGSKKVDDKRRKTVHNTKSRAKYS

SEQ ID NO: 178 - protein, 27 aa, artificial sequence
  Calcium carbonate binding peptide
RNNKGSKKVDDKRRKTVHSTKSRAKYS

SEQ ID NO: 179 - protein, 27 aa, artificial sequence
  Calcium carbonate binding peptide
RNNKGSRKVDDKRRKTVHNTKSRAKYS

SEQ ID NO: 180 - protein, 27 aa, artificial sequence
  Calcium carbonate binding peptide
RNNKGSKKADDKRRKTVHSTKSRAKYS

SEQ ID NO: 181 - protein, 27 aa, artificial sequence
  Calcium carbonate binding peptide
RNNKGSKKVDDKRRKAVHNKKSRAKYS

SEQ ID NO: 182 - protein, 27 aa, artificial sequence
  Calcium carbonate binding peptide
RNNKGSKKVDDKRRKTVHNTRSRAKYS

SEQ ID NO: 183 - protein, 27 aa, artificial sequence
  Calcium carbonate binding peptide
RNNKGSKKVDDKRRKTVHNTKSRAKFS

SEQ ID NO: 184 - protein, 27 aa, artificial sequence
  Calcium carbonate binding peptide
QRRKLRHPKEKWFGWSEKKVIKKWSRK

SEQ ID NO: 185 - protein, 27 aa, artificial sequence
  Calcium carbonate binding peptide
QRRKFRHPKEKWFGWSEKKVIKXNGRP

SEQ ID NO: 186 - protein, 27 aa, artificial sequence
  Calcium carbonate binding peptide
HKRLVQNKPHRTRKIEGWIKHMVKRQH

SEQ ID NO: 187 - protein, 27 aa, artificial sequence
  Calcium carbonate binding peptide
TRGHIMRPCWIGAMKQGVKKKRTPGWR

SEQ ID NO: 188 - protein, 27 aa, artificial sequence
  Calcium carbonate binding peptide
WKVKRRMVTRTYEFMGKKPCMMLTKRL

SEQ ID NO: 189 - protein, 26 aa, artificial sequence
  Calcium carbonate binding peptide
KKSNKGHHSKAKQKRPHGGKAQNKNT

SEQ ID NO: 190 - protein, 27 aa, artificial sequence
  Calcium carbonate binding peptide
RAHKERFVVRQIGRSQGYKTWQCVRVA

SEQ ID NO: 191 - protein, 27 aa, artificial sequence
  Calcium carbonate binding peptide
SQKPKGHKVKVVVKLCKRPYWRMLNTA

SEQ ID NO: 192 - protein, 27 aa, artificial sequence
  Calcium carbonate binding peptide
NHGCPVNWKVXNPPRGWQRLNHCKWWN

SEQ ID NO: 193 - protein, 27 aa, artificial sequence
  Calcium carbonate binding peptide
RNSRHKEWRRYKRTHVHSHEFYHVECW

SEQ ID NO: 194 - protein, 27 aa, artificial sequence
  Calcium carbonate binding peptide
HRSEKPKNVNYKRGYWERGNQKKHGPG

SEQ ID NO: 195 - protein, 27 aa, artificial sequence
  Calcium carbonate binding peptide
HERTRRGKPDRQKTTHEKRRQGLWIFM

SEQ ID NO: 196 - protein, 27 aa, artificial sequence
  Calcium carbonate binding peptide
PWGTNKRQKHKVHEAKALKKSLWYSNS

SEQ ID NO: 197 - protein, 27 aa, artificial sequence
  Calcium carbonate binding peptide
RRGVVLCHTHRNKRIRLAYSVTKKAWA

SEQ ID NO: 198 - protein, 27 aa, artificial sequence
  Calcium carbonate binding peptide
ERIRWRRLSAEIRAHKWSVLKFRLSCM

SEQ ID NO: 199 - protein, 27 aa, artificial sequence
  Calcium carbonate binding peptide
KTKEKKKEVKLHKKSLSLVLLADLWRL

SEQ ID NO: 200 - protein, 27 aa, artificial sequence
  Calcium carbonate binding peptide
LGKKHKQHSKVGHGKLSTRFLRRSKLF

SEQ ID NO: 201 - protein, 20 aa, artificial sequence
  Silica-binding peptide
AEAKRHPVVPLHEQHGHHEL

SEQ ID NO: 202 - protein, 18 aa, artificial sequence
  Silica-binding peptide
APQTWNRPHPGHPNVHTR

SEQ ID NO: 203 - protein, 20 aa, artificial sequence
  Silica-binding peptide
ATTPPSGKAAAHSAARQKGN

SEQ ID NO: 204 - protein, 20 aa, artificial sequence
  Silica-binding peptide
DGRPDNPKHQQSYNRQLPRQ

SEQ ID NO: 205 - protein, 20 aa, artificial sequence
  Silica-binding peptide
DHNNRQHAVEVRENKTHTAR

SEQ ID NO: 206 - protein, 20 aa, artificial sequence
  Silica-binding peptide
GPEPRALNPKRHMDPATQIR

SEQ ID NO: 207 - protein, 15 aa, artificial sequence
  Silica-binding peptide
HDHHQTHNVLHGMKK

SEQ ID NO: 208 - protein, 18 aa, artificial sequence
  Silica-binding peptide
HHDRAEPRGMAATLAQTI

SEQ ID NO: 209 - protein, 20 aa, artificial sequence
  Silica-binding peptide
HHNHMTGADNPIFHNNTAHR

SEQ ID NO: 210 - protein, 18 aa, artificial sequence
  Silica-binding peptide
HNHAQMLRPEPTGISHKN

SEQ ID NO: 211 - protein, 20 aa, artificial sequence
  Silica-binding peptide
HTNDNGQSTPRRDPPAFQRK

SEQ ID NO: 212 - protein, 18 aa, artificial sequence
  Silica-binding peptide
HTNHHYDQKMHGPLPTPY

SEQ ID NO: 213 - protein, 20 aa, artificial sequence
  Silica-binding peptide
LNSMSDKHHGHQNTATRNQH

SEQ ID NO: 214 - protein, 20 aa, artificial sequence
  Silica-binding peptide
MHKPNNPDTHRSTPSPLGKS

SEQ ID NO: 215 - protein, 18 aa, artificial sequence
  Silica-binding peptide
NFPVYDTTHHGGHRSKLH

SEQ ID NO: 216 - protein, 20 aa, artificial sequence
  Silica-binding peptide
NVHPQSENTNTTRPHKSTQR

SEQ ID NO: 217 - protein, 18 aa, artificial sequence
  Silica-binding peptide
QHGMHSPNLGARMNATPH

SEQ ID NO: 218 - protein, 20 aa, artificial sequence
  Silica-binding peptide
RPNDTHHPGKCDTHAVCHQT

SEQ ID NO: 219 - protein, 20 aa, artificial sequence
  Silica-binding peptide
SHLMHVKAPTDQASTRNRFD

SEQ ID NO: 220 - protein, 20 aa, artificial sequence
  Silica-binding peptide
SSSTPPNSPKHSKYNVWTSP

SEQ ID NO: 221 - protein, 17 aa, artificial sequence
  Silica-binding peptide
VHQTTPQHKDAVNLPRK

SEQ ID NO: 222 - protein, 20 aa, artificial sequence
  Silica-binding peptide
WHSSEGQYKKPNNHRQYHTG

SEQ ID NO: 223 - protein, 15 aa, artificial sequence
  Silica-binding peptide
YKHERHYSQPLKVRH

SEQ ID NO: 224 - protein, 17 aa, Artificial Sequence
  synthetic construct
PKGLKKLLKGLKKLLKL

SEQ ID NO: 225 - protein, 16 aa, Artificial Sequence
  synthetic construct
KGLKKLLKGLKKLLKL

SEQ ID NO: 226 - protein, 16 aa, Artificial Sequence
  synthetic construct
KGLKKLLKLLKKLLKL

SEQ ID NO: 227 - protein, 14 aa, Artificial Sequence
  synthetic construct
LKKLLKLLKKLLKL

SEQ ID NO: 228 - protein, 12 aa, Artificial Sequence
  synthetic construct
LKKLLKLLKKLL

SEQ ID NO: 229 - protein, 17 aa, Artificial Sequence
  synthetic construct
VAKKLAKLAKKLAKLAL

SEQ ID NO: 230 - protein, 13 aa, Artificial Sequence
  synthetic construct
FAKLLAKALKKLL

SEQ ID NO: 231 - protein, 16 aa, Artificial Sequence
  synthetic construct
KGLKKGLKLLKKLLKL

SEQ ID NO: 232 - protein, 16 aa, Artificial Sequence
  synthetic construct
KGLKKLLKLGKKLLKL

SEQ ID NO: 233 - protein, 16 aa, Artificial Sequence
  synthetic construct
KGLKKLGKLLKKLLKL

SEQ ID NO: 234 - protein, 16 aa, Artificial Sequence
  synthetic construct
KGLKKLLKLLKKGLKL

SEQ ID NO: 235 - protein, 16 aa, Artificial Sequence
  synthetic construct
KGLKKLLKLLKKLGKL

SEQ ID NO: 236 - protein, 19 aa, Artificial Sequence
  synthetic construct
FALALKALKKLKKALKKAL

SEQ ID NO: 237 - protein, 17 aa, Artificial Sequence
  synthetic construct
FAKKLAKLAKKLAKLAL

SEQ ID NO: 238 - protein, 13 aa, Artificial Sequence
  synthetic construct
FAKLLAKLAKKLL

SEQ ID NO: 239 - protein, 15 aa, Artificial Sequence
  synthetic construct
FAKKLAKLALKLAKL

SEQ ID NO: 240 - protein, 10 aa, Artificial Sequence
  synthetic construct
FAKKLAKKLL

SEQ ID NO: 241 - protein, 13 aa, Artificial Sequence
  synthetic construct
FAKLLAKLAKKVL

SEQ ID NO: 242 - protein, 13 aa, Artificial Sequence
  synthetic construct
KYKKALKKLAKLL

SEQ ID NO: 243 - protein, 12 aa, Artificial Sequence
  synthetic construct
FALLKALLKKAL

SEQ ID NO: 244 - protein, 14 aa, Artificial Sequence
  synthetic construct
KRLFKKLKFSLRKY

SEQ ID NO: 245 - protein, 14 aa, Artificial Sequence
  synthetic construct
KRLFKKLLFSLRKY

SEQ ID NO: 246 - protein, 14 aa, Artificial Sequence
  synthetic construct
LLLFLLKKRKKRKY

SEQ ID NO: 247 - protein, 36 aa, Hyalophora cecropia
KWKLFKKIEKVGQNIRDGIIKAGPAVAWGQATQIAK

SEQ ID NO: 248 - protein, 23 aa, Xenopus laevis
GIGKFLHSAKKFGKAFVGEIMNS

SEQ ID NO: 249 - protein, 22 aa, Xenopus laevis
GIGKFLKKAKKFGKAFVKILKK

SEQ ID NO: 250 - protein, 12 aa, Bos taurus
RLCRIVVIRVCR

SEQ ID NO: 251 - protein, 13 aa, Bos taurus
ILPWKWPWWPWRR

SEQ ID NO: 252 - protein, 24 aa, Homo sapiens
DSHAKRHHGYKRKFHEKHHSHRGY

SEQ ID NO: 253 - protein, 33 aa, artificial sequence
  synthetic linker
EPEPEPEPIPEPPKEAPVVIEKPKPKPKPKPKP

SEQ ID NO: 254 - protein, 21 aa, artificial sequence
  synthetic peptide linker
GKGKGKGKGKGKGKGKGKGKG

SEQ ID NO: 255 - protein, 8 aa, artificial sequence
  synthetic peptide bridge
GSGGGGSP

SEQ ID NO: 256 - protein, 16 aa, artificial sequence
  synthetic peptide bridge
GSGGGGSPGSGGGGSP

SEQ ID NO: 257 - protein, 28 aa, Artificial sequence
  PMMA-binding peptide
GWQRIWQSILCWMYFPLCLWMEWYRAIK

SEQ ID NO: 258 - protein, 12 aa, artificial sequence
  synthetic hair-binding peptide
RVPNKTVTVDGA

SEQ ID NO: 259 - protein, 12 aa, artificial sequence
  Synthetic construct
DRHKSKYSSTKS

SEQ ID NO: 260 - protein, 12 aa, artificial sequence
  Synthetic construct
KNFPQQKEFPLS

SEQ ID NO: 261 - protein, 12 aa, artificial sequence
  Synthetic construct
QRNSPPAMSRRD

SEQ ID NO: 262 - protein, 12 aa, artificial sequence
  Synthetic construct
TRKPNMPHGQYL

SEQ ID NO: 263 - protein, 12 aa, artificial sequence
  Synthetic construct
KPPHLAKLPFTT

SEQ ID NO: 264 - protein, 12 aa, artificial sequence
  Synthetic construct
NKRPPTSHRIHA

SEQ ID NO: 265 - protein, 12 aa, artificial sequence
  Synthetic construct
NLPRYQPPCKPL

SEQ ID NO: 266 - protein, 12 aa, artificial sequence
  Synthetic construct
RPPWKKPIPPSE

SEQ ID NO: 267 - protein, 12 aa, artificial sequence
  Synthetic construct
RQRPKDHFFSRP

SEQ ID NO: 268 - protein, 12 aa, artificial sequence
  Synthetic construct
SVPNKXVTVDGX

SEQ ID NO: 269 - protein, 12 aa, artificial sequence
  Synthetic construct
TTKWRHRAPVSP

SEQ ID NO: 270 - protein, 12 aa, artificial sequence
  Synthetic construct
WLGKNRIKPRAS

SEQ ID NO: 271 - protein, 12 aa, artificial sequence
  Synthetic construct
SNFKTPLPLTQS

SEQ ID NO: 272 - protein, 12 aa, artificial sequence
  Synthetic construct
SVSVGMKPSPRP

SEQ ID NO: 273 - protein, 7 aa, artificial sequence
  Synthetic construct
DLHTVYH

SEQ ID NO: 274 - protein, 7 aa, artificial sequence
  Synthetic construct
HIKPPTR

SEQ ID NO: 275 - protein, 7 aa, artificial sequence
  Synthetic construct
HPVWPAI

SEQ ID NO: 276 - protein, 7 aa, artificial sequence
  Synthetic construct
MPLYYLQ

SEQ ID NO: 277 - protein, 26 aa, artificial sequence
  Synthetic construct
HLTVPWRGGGSAVPFYSHSQITLPNH

SEQ ID NO: 278 - protein, 41 aa, artificial sequence
  Synthetic construct
GPHDTSSGGVRPNLHHTSKKEKRENRKVPFYSHSVTSRGNV

SEQ ID NO: 279 - protein, 7 aa, artificial sequence
  Synthetic construct
KHPTYRQ

SEQ ID NO: 280 - protein, 7 aa, artificial sequence
  Synthetic construct
HPMSAPR

SEQ ID NO: 281 - protein, 7 aa, artificial sequence
  Synthetic construct
MPKYYLQ

SEQ ID NO: 282 - protein, 7 aa, artificial sequence
  Synthetic construct
MHAHSIA

SEQ ID NO: 283 - protein, 12 aa, artificial sequence
  Synthetic construct
AKPISQHLQRGS

SEQ ID NO: 284 - protein, 12 aa, artificial sequence
  Synthetic construct
APPTPAAASATT

SEQ ID NO: 285 - protein, 12 aa, artificial sequence
  Synthetic construct
DPTEGARRTIMT

SEQ ID NO: 286 - protein, 12 aa, artificial sequence
  Synthetic construct
LDTSFPPVPFHA

SEQ ID NO: 287 - protein, 12 aa, artificial sequence
  Synthetic construct
LDTSFHQVPFHQ

SEQ ID NO: 288 - protein, 11 aa, artificial sequence
  Synthetic construct
LPRIANTWSPS

SEQ ID NO: 289 - protein, 12 aa, artificial sequence
  Synthetic construct
RTNAADHPAAVT

SEQ ID NO: 290 - protein, 12 aa, artificial sequence
  Synthetic construct
SLNWVTIPGPKI

SEQ ID NO: 291 - protein, 12 aa, artificial sequence
  Synthetic construct
TDMQAPTKSYSN

SEQ ID NO: 292 - protein, 12 aa, artificial sequence
  Synthetic construct
TIMTKSPSLSCG

SEQ ID NO: 293 - protein, 12 aa, artificial sequence
  Synthetic construct
TPALDGLRQPLR

SEQ ID NO: 294 - protein, 12 aa, artificial sequence
  Synthetic construct
TYPASRLPLLAP

SEQ ID NO: 295 - protein, 12 aa, artificial sequence
  Synthetic construct
AKTHKHPAPSYS

SEQ ID NO: 296 - protein, 12 aa, artificial sequence
  Synthetic construct
TDPTPFSISPER

SEQ ID NO: 297 - protein, 12 aa, artificial sequence
  Synthetic construct
SQNWQDSTSYSN

SEQ ID NO: 298 - protein, 12 aa, artificial sequence
  Synthetic construct
WHDKPQNSSKST

SEQ ID NO: 299 - protein, 12 aa, artificial sequence
  Synthetic construct
LDVESYKGTSMP

SEQ ID NO: 300 - protein, 7 aa, artificial sequence
  Synthetic construct
NTPKENW

SEQ ID NO: 301 - protein, 7 aa, artificial sequence
  Synthetic construct
NTPASNR

SEQ ID NO: 302 - protein, 7 aa, artificial sequence
  Synthetic construct
PRGMLST

SEQ ID NO: 303 - protein, 7 aa, artificial sequence
  Synthetic construct
PPTYLST

SEQ ID NO: 304 - protein, 12 aa, artificial sequence
  Synthetic construct
TIPTHRQHDYRS

SEQ ID NO: 305 - protein, 7 aa, artificial sequence
  Synthetic construct
TPPTHRL

SEQ ID NO: 306 - protein, 7 aa, artificial sequence
  Synthetic construct
LPTMSTP

SEQ ID NO: 307 - protein, 7 aa, artificial sequence
  Synthetic construct
LGTNSTP

SEQ ID NO: 308 - protein, 12 aa, artificial sequence
  Synthetic construct
TPLTGSTNLLSS

SEQ ID NO: 309 - protein, 7 aa, artificial sequence
  Synthetic construct
TPLTKET

SEQ ID NO: 310 - protein, 7 aa, artificial sequence
  Synthetic construct
KQSHNPP

SEQ ID NO: 311 - protein, 7 aa, artificial sequence
  Synthetic construct
QQSHNPP

SEQ ID NO: 312 - protein, 7 aa, artificial sequence
  Synthetic construct
TQPHNPP

SEQ ID NO: 313 - protein, 12 aa, artificial sequence
  Synthetic construct
STNLLRTSTVHP

SEQ ID NO: 314 - protein, 12 aa, artificial sequence
  Synthetic construct
HTQPSYSSTNLF

SEQ ID NO: 315 - protein, 7 aa, artificial sequence
  Synthetic construct
SLLSSHA

SEQ ID NO: 316 - protein, 12 aa, artificial sequence
  Synthetic construct
QQSSISLSSHAV

SEQ ID NO: 317 - protein, 7 aa, artificial sequence
  Synthetic construct
NASPSSL

SEQ ID NO: 318 - protein, 7 aa, artificial sequence
  Synthetic construct
HSPSSLR

SEQ ID NO: 319 - protein, 7 aa, artificial sequence
  Synthetic construct
KXSHHTH

SEQ ID NO: 320 - protein, 7 aa, artificial sequence
  Synthetic construct
EXSHHTH

SEQ ID NO: 321 - protein, 12 aa, artificial sequence
  Synthetic construct
SHHTHYGQPGPV

SEQ ID NO: 322 - protein, 7 aa, artificial sequence
  Synthetic construct
LESTSLL

SEQ ID NO: 323 - protein, 7 aa, artificial sequence
  Synthetic construct
DLTLPFH

SEQ ID NO: 324 - protein, 8 aa, artificial sequence
  Synthetic construct
RTNAADHP

SEQ ID NO: 325 - protein, 12 aa, artificial sequence
  Synthetic construct
IPWWNIRAPLNA

SEQ ID NO: 326 - protein, 18 aa, artificial sequence
  Synthetic construct
EQISGSLVAAPWEGEGER

SEQ ID NO: 327 - protein, 12 aa, artificial sequence
  synthetic hair-binding peptide
TPPELLHGAPRS

SEQ ID NO: 328 - protein, 18 aa, artificial sequence
  Synthetic construct
LDTSFHQVPFHQKRKRKD

SEQ ID NO: 329 - protein, 18 aa, artificial sequence
  Synthetic construct
EQISGSLVAAPWKRKRKD

SEQ ID NO: 330 - protein, 18 aa, artificial sequence
  Synthetic construct
TPPELLHGDPRSKRKRKD

SEQ ID NO: 331 - protein, 13 aa, artificial sequence
  Synthetic construct
NTSQLSTEGEGED

SEQ ID NO: 332 - protein, 13 aa, artificial sequence
  Synthetic construct
TPPELLHGDPRSC

SEQ ID NO: 333 - protein, 20 aa, artificial sequence
  synthetic hair-binding peptide
HINKTNPHQGNHHSEKTQRQ

SEQ ID NO: 334 - protein, 15 aa, artificial sequence
  Synthetic construct
HAHKNQKETHQRHAA

SEQ ID NO: 335 - protein, 15 aa, artificial sequence
  Synthetic construct
HEHKNQKETHQRHAA

SEQ ID NO: 336 - protein, 20 aa, artificial sequence
  Synthetic construct
HNHMQERYTEPQHSPSVNGL

SEQ ID NO: 337 - protein, 17 aa, artificial sequence
  Synthetic construct
THSTHNHGSPRHTNADA

SEQ ID NO: 338 - protein, 20 aa, artificial sequence
  synthetic hair-binding peptide
GSCVDTHKADSCVANNGPAT

SEQ ID NO: 339 - protein, 20 aa, artificial sequence
  synthetic hair-binding peptide
AQSQLPDKHSGLHERAPQRY

SEQ ID NO: 340 - protein, 20 aa, artificial sequence
  Synthetic construct
AQSQLPAKHSGLHERAPQRY

SEQ ID NO: 341 - protein, 20 aa, artificial sequence
  Synthetic construct
AQSQLPEKHSGLHERAPQRY

SEQ ID NO: 342 - protein, 20 aa, artificial sequence
  synthetic hair-binding peptide
TDMMHNHSDNSPPHRRSPRN

SEQ ID NO: 343 - protein, 20 aa, artificial sequence
  synthetic hair-binding peptide
TPPELAHTPHHLAQTRLTDR

SEQ ID NO: 344 - protein, 12 aa, artificial sequence
  Synthetic construct
RLLRLLRLLRLL

SEQ ID NO: 345 - protein, 12 aa, artificial sequence
  Synthetic construct
TPPELLHGEPRS

SEQ ID NO: 346 - protein, 12 aa, artificial sequence
  Synthetic construct
TPPELLHGAPRS

SEQ ID NO: 347 - protein, 12 aa, artificial sequence
  Synthetic construct
EQISGSLVAAPW

SEQ ID NO: 348 - protein, 12 aa, artificial sequence
  Synthetic construct
NEVPARNAPWLV

SEQ ID NO: 349 - protein, 13 aa, artificial sequence
  Synthetic construct
NSPGYQADSVAIG

SEQ ID NO: 350 - protein, 12 aa, artificial sequence
  Synthetic construct
AKPISQHLQRGS

SEQ ID NO: 351 - protein, 12 aa, artificial sequence
  Synthetic construct
LDTSFPPVPFHA

SEQ ID NO: 352 - protein, 12 aa, artificial sequence
  Synthetic construct
SLNWVTIPGPKI

SEQ ID NO: 353 - protein, 12 aa, artificial sequence
  Synthetic construct
TQDSAQKSPSPL

SEQ ID NO: 354 - protein, 12 aa, artificial sequence
  Synthetic construct
KELQTRNVVQRE

SEQ ID NO: 355 - protein, 12 aa, artificial sequence
  Synthetic construct
QRNSPPAMSRRD

SEQ ID NO: 356 - protein, 12 aa, artificial sequence
  Synthetic construct
TPTANQFTQSVP

SEQ ID NO: 357 - protein, 12 aa, artificial sequence
  Synthetic construct
AAGLSQKHERNR

SEQ ID NO: 358 - protein, 12 aa, artificial sequence
  Synthetic construct
ETVHQTPLSDRP

SEQ ID NO: 359 - protein, 12 aa, artificial sequence
  Synthetic construct
KNFPQQKEFPLS

SEQ ID NO: 360 - protein, 12 aa, artificial sequence
  Synthetic construct
LPALHIQRHPRM

SEQ ID NO: 361 - protein, 12 aa, artificial sequence
  Synthetic construct
QPSHSQSHNLRS

SEQ ID NO: 362 - protein, 12 aa, artificial sequence
  Synthetic construct
RGSQKSKPPRPP

SEQ ID NO: 363 - protein, 12 aa, artificial sequence
  Synthetic construct
THTQKTPLLYYH

SEQ ID NO: 364 - protein, 12 aa, artificial sequence
  Synthetic construct
TKGSSQAILKST

SEQ ID NO: 365 - protein, 7 aa, artificial sequence
  Synthetic construct
TAATTSP

SEQ ID NO: 366 - protein, 7 aa, artificial sequence
  Synthetic construct
LGIPQNL

SEQ ID NO: 367 - protein, 20 aa, artificial sequence
  Synthetic construct
THSTHNHGSPRHTNADAGNP

SEQ ID NO: 368 - protein, 20 aa, artificial sequence
  Synthetic construct
QQHKVHHQNPDRSTQDAHHS

SEQ ID NO: 369 - protein, 15 aa, artificial sequence
  Synthetic construct
HHGTHHNATKQKNHV

SEQ ID NO: 370 - protein, 15 aa, artificial sequence
  Synthetic construct
STLHKYKSQDPTPHH

SEQ ID NO: 371 - protein, 12 aa, artificial sequence
  Synthetic construct
SVSVGMKPSPRP

SEQ ID NO: 372 - protein, 12 aa, artificial sequence
  synthetic hair-binding peptide
TPPTNVLMLATK

SEQ ID NO: 373 - protein, 12 aa, artificial sequence
  Synthetic construct
TPPELLHGDPRS

SEQ ID NO: 374 - protein, 7 aa, artificial sequence
  synthetic hair-binding peptide
NTSQLST

SEQ ID NO: 375 - protein, 15 aa, artificial sequence
  Synthetic construct
STLHKYKSQDPTPHH

SEQ ID NO: 376 - protein, 12 aa, artificial sequence
  synthetic hair-binding peptide
GMPAMHWIHPFA

SEQ ID NO: 377 - protein, 15 aa, artificial sequence
  synthetic hair-binding peptide
HDHKNQKETHQRHAA

SEQ ID NO: 378 - protein, 20 aa, artificial sequence
  Synthetic construct
HNHMQERYTDPQHSPSVNGL

SEQ ID NO: 379 - protein, 20 aa, artificial sequence
  synthetic hair-binding peptide
TAEIQSSKNPNPHPQRSWTN

SEQ ID NO: 380 - protein, 12 aa, artificial sequence
  Synthetic construct
KRGRHKRPKRHK

SEQ ID NO: 381 - protein, 7 aa, artificial sequence
  Synthetic construct
RLLRLLR

SEQ ID NO: 382 - protein, 12 aa, artificial sequence
  Synthetic construct
HKPRGGRKKALH

SEQ ID NO: 383 - protein, 18 aa, artificial sequence
  Synthetic construct
KPRPPHGKKHRPKHRPKK

SEQ ID NO: 384 - protein, 18 aa, artificial sequence
  Synthetic construct
RGRPKKGHGKRPGHRARK

SEQ ID NO: 385 - protein, 12 aa, artificial sequence
  Synthetic construct
TPFHSPENAPGS

SEQ ID NO: 386 - protein, 13 aa, artificial sequence
  Synthetic construct
TPFHSPENAPGSK

SEQ ID NO: 387 - protein, 16 aa, artificial sequence
  Synthetic construct
TPFHSPENAPGSGGGS

SEQ ID NO: 388 - protein, 17 aa, artificial sequence
  Synthetic construct
TPFHSPENAPGSGGGSS

SEQ ID NO: 389 - protein, 15 aa, artificial sequence
  Synthetic construct
TPFHSPENAPGSGGG

SEQ ID NO: 390 - protein, 7 aa, artificial sequence
  Synthetic construct
FTQSLPR

SEQ ID NO: 391 - protein, 12 aa, artificial sequence
  Synthetic construct
KQATFPPNPTAY

SEQ ID NO: 392 - protein, 12 aa, artificial sequence
  Synthetic construct
HGHMVSTSQLSI

SEQ ID NO: 393 - protein, 7 aa, artificial sequence
  Synthetic construct
LSPSRMK

SEQ ID NO: 394 - protein, 7 aa, artificial sequence
  Synthetic construct
LPIPRMK

SEQ ID NO: 395 - protein, 7 aa, artificial sequence
  Synthetic construct
HQRPYLT

SEQ ID NO: 396 - protein, 7 aa, artificial sequence
  Synthetic construct
FPPLLRL

SEQ ID NO: 397 - protein, 7 aa, artificial sequence
  Synthetic construct
QATFMYN

SEQ ID NO: 398 - protein, 11 aa, artificial sequence
  Synthetic construct
VLTSQLPNHSM

SEQ ID NO: 399 - protein, 7 aa, artificial sequence
  Synthetic construct
HSTAYLT

SEQ ID NO: 400 - protein, 12 aa, artificial sequence
  Synthetic construct
APQQRPMKTFNT

SEQ ID NO: 401 - protein, 12 aa, artificial sequence
  Synthetic construct
APQQRPMKTVQY

SEQ ID NO: 402 - protein, 7 aa, artificial sequence
  Synthetic construct
PPWLDLL

SEQ ID NO: 403 - protein, 7 aa, artificial sequence
  Synthetic construct
PPWTFPL

SEQ ID NO: 404 - protein, 7 aa, artificial sequence
  Synthetic construct
SVTHLTS

SEQ ID NO: 405 - protein, 7 aa, artificial sequence
  Synthetic construct
VITRLTS

SEQ ID NO: 406 - protein, 12 aa, artificial sequence
  Synthetic construct
DLKPPLLALSKV

SEQ ID NO: 407 - protein, 12 aa, artificial sequence
  Synthetic construct
SHPSGALQEGTF

SEQ ID NO: 408 - protein, 12 aa, artificial sequence
  Synthetic construct
FPLTSKPSGACT

SEQ ID NO: 409 - protein, 12 aa, artificial sequence
  Synthetic construct
DLKPPLLALSKV

SEQ ID NO: 410 - protein, 7 aa, artificial sequence
  Synthetic construct
PLLALHS

SEQ ID NO: 411 - protein, 7 aa, artificial sequence
  Synthetic construct
VPISTQI

SEQ ID NO: 412 - protein, 12 aa, artificial sequence
  Synthetic construct
YAKQHYPISTFK

SEQ ID NO: 413 - protein, 7 aa, artificial sequence
  Synthetic construct
HSTAYLT

SEQ ID NO: 414 - protein, 12 aa, artificial sequence
  Synthetic construct
STAYLVAMSAAP

SEQ ID NO: 415 - protein, 12 aa, artificial sequence
  Synthetic construct
SVSVGMKPSPRP

SEQ ID NO: 416 - protein, 12 aa, artificial sequence
  Synthetic construct
TMGFTAPRFPHY

SEQ ID NO: 417 - protein, 12 aa, artificial sequence
  Synthetic construct
NLQHSVGTSPVW

SEQ ID NO: 418 - protein, 15 aa, artificial sequence
  Synthetic construct
QLSYHAYPQANHHAP

SEQ ID NO: 419 - protein, 12 aa, artificial sequence
  Synthetic construct
NQAASITKRVPY

SEQ ID NO: 420 - protein, 14 aa, artificial sequence
  Synthetic construct
SGCHLVYDNGFCDH

SEQ ID NO: 421 - protein, 14 aa, artificial sequence
  Synthetic construct
ASCPSASHADPCAH

SEQ ID NO: 422 - protein, 14 aa, artificial sequence
  Synthetic construct
NLCDSARDSPRCKV

SEQ ID NO: 423 - protein, 12 aa, artificial sequence
  Synthetic construct
NHSNWKTAADFL

SEQ ID NO: 424 - protein, 12 aa, artificial sequence
  Synthetic construct
GSSTVGRPLSYE

SEQ ID NO: 425 - protein, 12 aa, artificial sequence
  Synthetic construct
SDTISRLHVSMT

SEQ ID NO: 426 - protein, 12 aa, artificial sequence
  Synthetic construct
SPLTVPYERKLL

SEQ ID NO: 427 - protein, 12 aa, artificial sequence
  Synthetic construct
SPYPSWSTPAGR

SEQ ID NO: 428 - protein, 12 aa, artificial sequence
  Synthetic construct
VQPITNTRYEGG

SEQ ID NO: 429 - protein, 12 aa, artificial sequence
  Synthetic construct
WPMHPEKGSRWS

SEQ ID NO: 430 - protein, 14 aa, artificial sequence
  Synthetic construct
DACSGNGHPNNCDR

SEQ ID NO: 431 - protein, 14 aa, artificial sequence
  Synthetic construct
DHCLGRQLQPVCYP

SEQ ID NO: 432 - protein, 14 aa, artificial sequence
  Synthetic construct
DWCDTIIPGRTCHG

SEQ ID NO: 433 - protein, 12 aa, artificial sequence
  Synthetic construct
ALPRIANTWSPS

SEQ ID NO: 434 - protein, 12 aa, artificial sequence
  Synthetic construct
YPSFSPTYRPAF

SEQ ID NO: 435 - protein, 20 aa, artificial sequence
  Synthetic construct
AHPESLGIKYALDGNSDPHA

SEQ ID NO: 436 - protein, 20 aa, artificial sequence
  Synthetic construct
ASVSNYPPIHHLATSNTTVN

SEQ ID NO: 437 - protein, 14 aa, artificial sequence
  Synthetic construct
DECMEPLNAAHCWR

SEQ ID NO: 438 - protein, 14 aa, artificial sequence
  Synthetic construct
DECMHGSDVEFCTS

SEQ ID NO: 439 - protein, 14 aa, artificial sequence
  Synthetic construct
DLCSMQMMNTGCHY

SEQ ID NO: 440 - protein, 14 aa, artificial sequence
  Synthetic construct
DLCSSPSTWGSCIR

SEQ ID NO: 441 - protein, 20 aa, artificial sequence
  Synthetic construct
DPNESNYENATTVSQPTRHL

SEQ ID NO: 442 - protein, 20 aa, artificial sequence
  Synthetic construct
EPTHPTMRAQMHQSLRSSSP

SEQ ID NO: 443 - protein, 20 aa, artificial sequence
  Synthetic construct
GNTDTTPPNAVMEPTVQHKW

SEQ ID NO: 444 - protein, 15 aa, artificial sequence
  Synthetic construct
NGPDMVQSVGKHKNS

SEQ ID NO: 445 - protein, 15 aa, artificial sequence
  Synthetic construct
NGPEVRQIPANFEKL

SEQ ID NO: 446 - protein, 20 aa, artificial sequence
  Synthetic construct
NNTSADNPPETDSKHHLSMS

SEQ ID NO: 447 - protein, 20 aa, artificial sequence
  Synthetic construct
NNTWPEGAGHTMPSTNIRQA

SEQ ID NO: 448 - protein, 20 aa, artificial sequence
  Synthetic construct
NPTATPHMKDPMHSNAHSSA

SEQ ID NO: 449 - protein, 20 aa, artificial sequence
  Synthetic construct
NPTDHIPANSTNSRVSKGNT

SEQ ID NO: 450 - protein, 15 aa, artificial sequence
  Synthetic construct
NPTDSTHMMHARNHE

SEQ ID NO: 451 - protein, 14 aa, artificial sequence
  Synthetic construct
QHCITERLHPPCTK

SEQ ID NO: 452 - protein, 14 aa, artificial sequence
  Synthetic construct
TPCAPASFNPHCSR

SEQ ID NO: 453 - protein, 14 aa, artificial sequence
  Synthetic construct
TPCATYPHFSGCRA

SEQ ID NO: 454 - protein, 20 aa, artificial sequence
  Synthetic construct
WCTDFCTRSTPTSTSRSTTS

SEQ ID NO: 455 - protein, 20 aa, artificial sequence
  Synthetic construct
APPLKTYMQERELTMSQNKD

SEQ ID NO: 456 - protein, 20 aa, artificial sequence
  Synthetic construct
EPPTRTRVNNHTVTVQAQQH

SEQ ID NO: 457 - protein, 14 aa, artificial sequence
  Synthetic construct
GYCLRGDEPAVCSG

SEQ ID NO: 458 - protein, 20 aa, artificial sequence
  Synthetic construct
LSSKDFGVTNTDQRTYDYTT

SEQ ID NO: 459 - protein, 14 aa, artificial sequence
  Synthetic construct
NFCETQLDLSVCTV

SEQ ID NO: 460 - protein, 14 aa, artificial sequence
  Synthetic construct
NTCQPTKNATPCSA

SEQ ID NO: 461 - protein, 20 aa, artificial sequence
  Synthetic construct
PSEPERRDRNIAANAGRFNT

SEQ ID NO: 462 - protein, 18 aa, artificial sequence
  Synthetic construct
THNMSHFPPSGHPKRTAT

SEQ ID NO: 463 - protein, 14 aa, artificial sequence
  Synthetic construct
TTCPTMGTYHVCWL

SEQ ID NO: 464 - protein, 20 aa, artificial sequence
  Synthetic construct
YCADHTPDPANPNKICGYSH

SEQ ID NO: 465 - protein, 20 aa, artificial sequence
  Synthetic construct
AANPHTEWDRDAFQLAMPPK

SEQ ID NO: 466 - protein, 20 aa, artificial sequence
  Synthetic construct
DLHPMDPSNKRPDNPSDLHT

SEQ ID NO: 467 - protein, 14 aa, artificial sequence
  Synthetic construct
ESCVSNALMNQCIY

SEQ ID NO: 468 - protein, 20 aa, artificial sequence
  Synthetic construct
HNKADSWDPDLPPHAGMSLG

SEQ ID NO: 469 - protein, 20 aa, artificial sequence
  Synthetic construct
LNDQRKPGPPTMPTHSPAVG

SEQ ID NO: 470 - protein, 14 aa, artificial sequence
  Synthetic construct
NTCATSPNSYTCSN

SEQ ID NO: 471 - protein, 14 aa, artificial sequence
  Synthetic construct
SDCTAGLVPPLCAT

SEQ ID NO: 472 - protein, 20 aa, artificial sequence
  Synthetic construct
TIESSQHSRTHQQNYGSTKT

SEQ ID NO: 473 - protein, 20 aa, artificial sequence
  Synthetic construct
VGTMKQHPTTTQPPRVSATN

SEQ ID NO: 474 - protein, 20 aa, artificial sequence
  Synthetic construct
YSETPNDQKPNPHYKVSGTK